(19) United States
(12) Patent Application Publication
(10) Pub. No.: US 2023/0372095 A1
(43) Pub. Date: Nov. 23, 2023

(54) BALLOON EXPANDABLE TRANSCATHETER VALVE DELIVERY DEVICE SHAFT REINFORCEMENT DEVICES

(71) Applicant: Medtronic, Inc., Minneapolis, MN (US)

(72) Inventors: Reiss CONNOLLY, Limerick (IE); Niall DUFFY, Galway (IE); Jim PHELAN, Galway (IE); Constantin CIOBANU, Galway (IE); Marc ANDERSON, Galway (IE); Eoghan TWOHIG, Galway (IE); Gavin MOORE, Dublin (IE); Michael O'CONNOR, Galway (IE); Stephen MONTGOMERY, Galway (IE); Gerard COONEY, Dublin (IE); Micheal FALLON, Roscommon (IE); Huda KHILJI, Cavan (IE); David LYDON, Galway (IE); Alan MCGUINN, Oranmore (IE); Ciaran MCGUINNESS, Castlerea (IE); Conleth MULLEN, Galway (IE); Matthew NORGROVE, Dundrum (IE); Tomas KITT, Ballinasloe (IE)

(21) Appl. No.: 18/248,277

(22) PCT Filed: Oct. 7, 2021
(86) PCT No.: PCT/US2021/053942

§ 371 Date: Apr. 7, 2023

Related U.S. Application Data
(60) Provisional application No. 63/239,563, filed on Sep. 1, 2021.
(60) Provisional application No. 63/239,550, filed on Sep. 1, 2021.
(60) Provisional application No. 63/089,253, filed on Oct. 8, 2020.

(51) Int. Cl. A61F 2/24

(52) U.S. Cl. CPC A61F 2/2433

(57) ABSTRACT

A balloon catheter configured for delivery of a prosthetic heart valve is provided. The balloon catheter is configured to deploy a prosthetic heart valve through inflation. The balloon catheter is provided with a reinforcing member configured to increase the rigidity of the balloon catheter over an operational portion of the device. The increased rigidity serves to prevent damage to an introducer sheath, patient anatomy, or the prosthetic heart valve during delivery of the prosthetic heart valve.

### FIELD OF THE INVENTION

The present invention is related to systems and methods for transcatheter valve deployment. In particular, the present invention is related to balloon expandable transcatheter valve delivery devices configured for the deployment of prosthetic heart valves.

### BACKGROUND

Transcatheter prosthetic heart valve technology provides a minimally invasive means of implanting prosthetic heart valves. The prosthetic heart valve is loaded onto a delivery system that is able to access and navigate the vasculature to the intended implant location and implant the valve. A conventional approach for a transcatheter valve device is to use a balloon catheter for the delivery system and a prosthetic heart valve incorporating a balloon expandable frame. After reaching the delivery site, the balloon is inflated to expand the prosthetic heart valve into a deployment configuration. After deployment, the balloon is deflated and the balloon catheter is removed. When navigating a conventional balloon catheter delivery device through twists and turns of a patient's vasculature, particularly, the tight turn of the aortic arch presents, disparities in rigidity between the balloon catheter and the prosthetic heart valve crimped onto the balloon catheter can result in the protrusion of portions of the prosthetic heart valve. Such protrusions can result in damage to the valve, damage to the introducer sheath through which the delivery device is delivered, failed valve deployment, damage to the wall of the aorta, and dislodging of atheromas, plaque, etc., which may lead to an increased risk of stroke.

Devices and methods disclosed herein address the issue of prosthetic heart valve protrusions causing damage during valve delivery.

### SUMMARY

Embodiments of the present invention relate generally to delivery devices for prosthetic heart valves, and, more specifically, to balloon enabled prosthetic heart valve delivery devices. Balloon enabled prosthetic heart valve delivery devices consistent with embodiments hereof are configured to reduce or prevent prosthetic heart valve protrusion.

In an embodiment, a prosthetic heart valve stent having a crimped configuration for delivery within a vasculature and an expanded configuration for deployment within a native heart valve is provided. The prosthetic heart valve stent includes a central frame structure including a plurality of struts and a plurality of crowns; a first terminal frame structure extending from the central frame structure at a proximal end of the central frame structure, the first terminal frame structure including a first plurality of terminal struts and a first plurality of terminal crowns, the first plurality of terminal struts being connected to the central frame structure at a first plurality of angled crown junctions and the first plurality of crowns forming a first substantially non-zero crown junction angle with a linear projection of the central frame structure; a second terminal frame structure extending from the central frame structure at a distal end of the central frame structure, the second terminal frame structure including a second plurality of terminal struts and a second plurality of terminal crowns, the second plurality of terminal struts being connected to the central frame structure at a second plurality of angled crown junctions and the second plurality of crowns forming a second substantially non-zero crown junction angle with a linear projection of the central frame structure.

In another embodiment, a balloon catheter for deploying a prosthetic heart valve via balloon inflation is provided. The balloon catheter includes an inner shaft; an outer shaft surrounding the inner shaft; a balloon disposed at a distal end of the outer shaft defining a delivery portion of the balloon catheter; a prosthetic heart valve disposed over and crimped to the balloon, the prosthetic heart valve including a central frame structure and a plurality of terminal crowns extending from the central frame structure and disposed at a substantially non-zero crown junction angle with respect to the stent; a proximal retention bumper secured to the inner shaft proximal of the operational portion; a distal retention bumper secured to the inner shaft distal of the operational portion.

### BRIEF DESCRIPTION OF THE DRAWINGS

The accompanying figures, which are incorporated herein, form part of the specification and illustrate embodiments of a prosthesis delivery system. Together with the description, the figures further explain the principles of and enable a person skilled in the relevant art(s) to make and use the balloon catheters described herein. In the drawings, like reference numbers indicate identical or functionally similar elements.

FIG. 1 illustrates a balloon expandable frame of a prosthetic heart valve.

FIGS. 2A-2B illustrate an embodiment of a balloon catheter during a delivery operation.

FIGS. 3A-3B illustrate cross-sections of an embodiment of a balloon catheter during a delivery operation.

FIGS. 4A-4C illustrate an embodiment of a balloon catheter having a reinforcing member during a delivery operation.

FIGS. 5A-5D illustrate embodiments of reinforcing members consistent with embodiments hereof.

FIG. 6 illustrates an embodiment of reinforcing members consistent with embodiments hereof.

FIG. 7 illustrates an embodiment of reinforcing members consistent with embodiments hereof.

FIGS. 8A-8C illustrate embodiments of reinforcing members consistent with embodiments hereof.

FIG. 9 illustrates embodiments of retention bumpers consistent with embodiments hereof.

FIG. 10 illustrates an embodiment of reinforcing members consistent with embodiments hereof.

FIGS. 11A-11B illustrate embodiments of multipart retention bumpers consistent with embodiments hereof.

FIG. 12 illustrates an embodiment of reinforcing members consistent with embodiments hereof.

FIG. 13 illustrates a prosthetic heart valve consistent with embodiments hereof.

FIGS. 14A-14B illustrate a balloon catheter consistent with embodiments hereof.

FIG. 15 illustrates a portion of a balloon catheter featuring retention bumpers configured for use with prosthetic heart valves according to embodiments hereof.

FIG. 16 is a flow chart of a method of balloon deployment of a prosthetic heart valve consistent with embodiments described herein.

### DETAILED DESCRIPTION OF THE INVENTION

Specific embodiments of the present invention are now described with reference to the figures. Unless otherwise indicated, for the delivery devices, balloon catheters, and prosthetic heart valves described herein, the terms “distal” and “proximal” are used in the following description with respect to a position or direction relative to the treating clinician or operator. “Distal” and “distally” are positions distant from or in a direction away from the clinician, and “proximal” and “proximally” are positions near or in a direction toward the clinician.

The following detailed description is merely illustrative in nature and is not intended to limit the invention or the application and uses of the invention. Although the description of the invention is in the context of balloon enabled deployment of prosthetic heart valves, aspects of the invention may also be used in any other context that is useful. As an example, the description of the invention is in the context of deployment of prosthesis. As used herein, “prosthesis” or “prostheses” may include any prosthesis including a balloon expandable structure. Modifications can be made to the embodiments described herein without departing from the spirit and scope of the present invention. Therefore, the following detailed description is not meant to be limiting. Furthermore, there is no intention to be bound by any expressed or implied theory presented in the preceding technical field, background, summary, or the following detailed description.

Balloon catheters consistent with embodiments hereof are configured to deliver and deploy, through the use of an inflatable balloon, transcatheter balloon expandable prosthetic heart valves (referred to herein as “prosthetic heart valves”). Transcatheter balloon expandable prosthetic heart valves are crimped onto the balloon of the delivery system. The balloon of the delivery system is processed such that the balloon is pleated then folded prior to crimping the prosthetic heart valve. The prosthetic heart valve is deployed by inflating the balloon which thereby radially expands the prosthetic heart valve. Delivery of the prosthetic heart valve may be challenging due to the twists and turns of a patient's vascular anatomy. During delivery, disparities in rigidity between the prosthetic heart valve and the balloon catheter can cause disparities in bending of these parts. Such disparate bending can cause portions of the prosthetic heart valve, e.g., the crowns, to protrude from the path of the balloon catheter. Such protrusions can contact the introducer sheath, causing damage to the valve or the introducer sheath or causing movement of the valve relative to the balloon. In configurations where the portion of the balloon catheter carrying the prosthetic heart valve extends into the patient anatomy distal of the introducer sheath, the protrusions may directly contact the walls of the patient vasculature and thereby potentially cause damage. Each of these conditions may lead to a failed valve deployment. Embodiments hereof, as described below, provide selectively reinforced balloon delivery catheters which serve to reduce or prevent prosthetic heart valve protrusion during deployment.

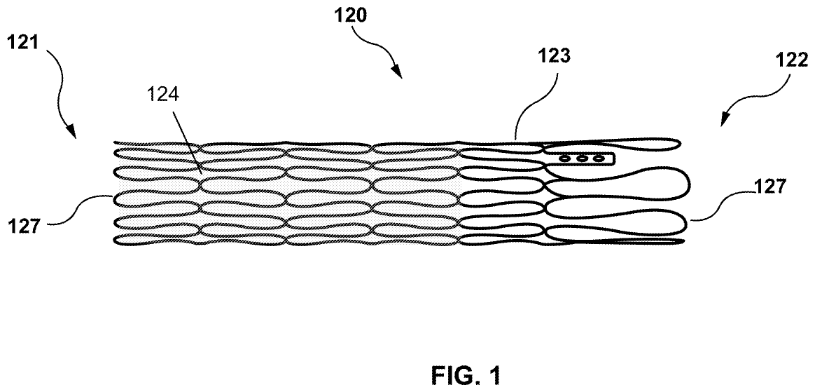

FIG. 1 illustrates a prosthetic heart valve 120 including its associated balloon expandable stent 123. The prosthetic heart valve 120 is configured for delivery by a balloon catheter. The prosthetic heart valve 120 includes a stent 123, graft material 124 disposed thereon, and a valve (not shown). The prosthetic heart valve 120 has a proximal end 122 and a distal end 121. The prosthetic heart valve 120 includes a plurality of crowns 127 located at the distal end 121 and at the proximal end 122. The crowns 127 are loops in the stent 123 of the prosthetic heart valve 120. The prosthetic heart valve 120 is non-symmetrical, having different expandable stent 123 geometry and construction at the proximal end 122 and the distal end 121. Other prosthetic heart valves may differ in design, having expandable frames that differ in structure and construction and prosthetic heart valve elements that also differ. Accordingly, different prosthetic heart valves may react differently in delivery conditions, due to variations in structural characteristics, such as radial stiffness. Because different prosthetic heart valves react differently to expansion, embodiments hereof are designed to prevent crown protrusion of prosthetic heart valves regardless of their structure and construction.

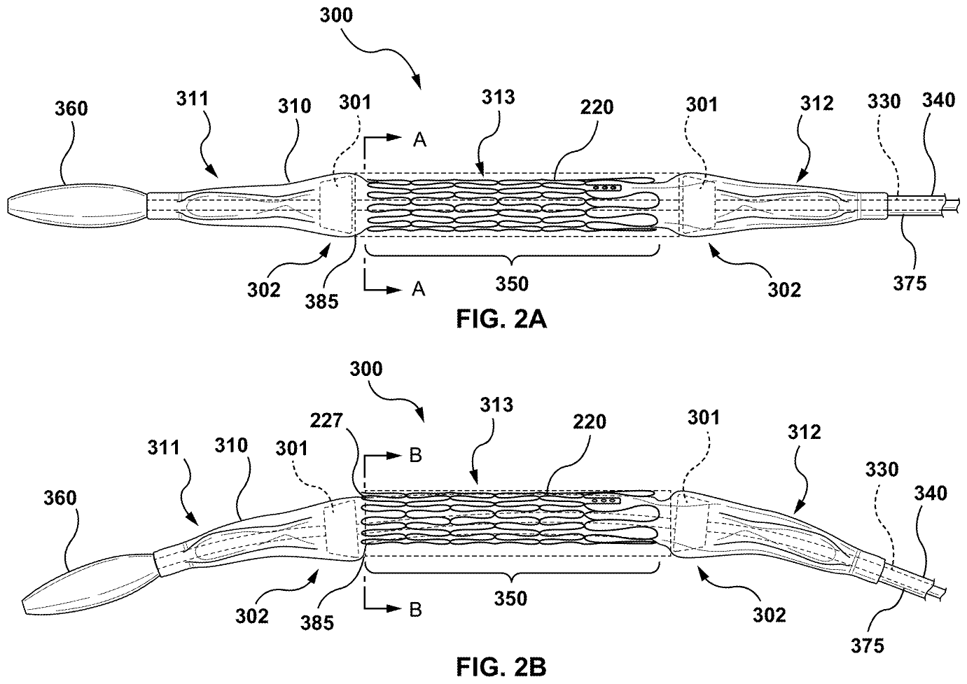

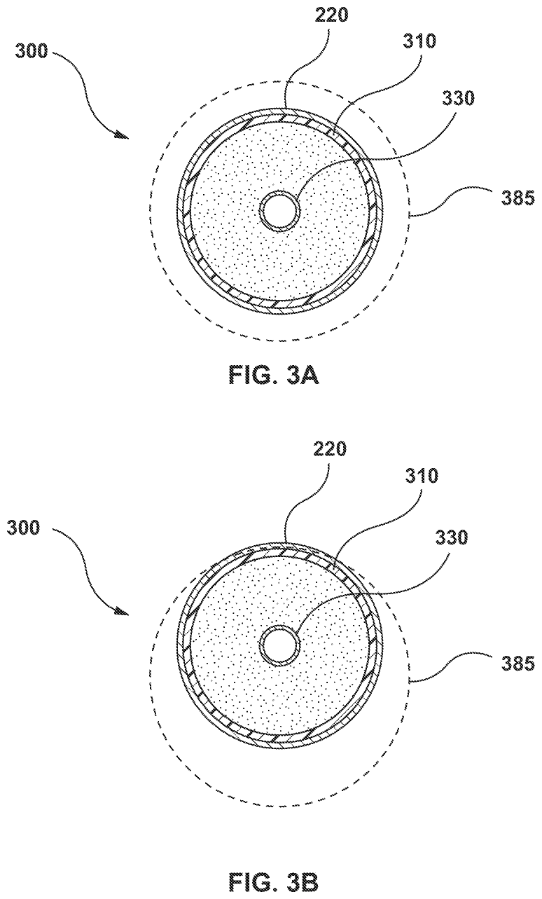

FIGS. 2-3 illustrate an expandable balloon delivery device, also referred to herein as a balloon catheter, and operation thereof. FIGS. 2-3 provide a detailed description of challenges encountered with balloon catheters that are not selectively reinforced.

FIGS. 2A and 2B illustrate a potential failure mode that may occur in some balloon catheters. Balloon catheter 300 includes an outer shaft 340, an inner shaft 330, a balloon 310, a distal tip 360, and a prosthetic heart valve 220. Inflation fluid is delivered to the balloon 310 via an inflation lumen 375 disposed between the outer shaft 340 and the inner shaft 330. The balloon 310 includes a distal portion 311, a proximal portion 312, and a central portion 313. The central portion 313 is the portion over which the prosthetic heart valve 220 is crimped. The central portion 313 of the balloon 310 coincides with an operational portion 350 of the balloon catheter 300. The operational portion 350 of the balloon catheter 300 refers to the portion of the balloon catheter 300 wherein the prosthetic heart valve 220 is disposed for deployment. The balloon catheter 300 further includes proximal and distal retention bumpers 301. The balloon catheter 300 may be operated in a similar fashion as other balloon catheters described herein, e.g., balloon catheter 300 as illustrated in FIGS. 2A-2B. The retention bumpers 301 may serve several purposes.

The retention bumpers 301 may function to maintain an axial position of the prosthetic heart valve 220. The retention bumpers 301 are positioned adjacent to the prosthetic heart valve 220 so as to maintain an axial position, i.e., prevent or reduce axial migration, through contact. As shown in FIG. 2A, a proximal retention bumper 301 is positioned adjacent the proximal end of the prosthetic heart valve 220 and the distal retention bumper 301 is positioned adjacent the distal end of the prosthetic heart valve 220. When subject to proximal or distal forces, the prosthetic heart valve 220 presses up against the portion of the balloon 310 raised by the retention bumpers 301, which arrests the movement of the prosthetic heart valve 220.

The retention bumpers 301 may also function to reduce forces acting on the prosthetic heart valve 220. The retention bumpers are sized such that they (or the balloon 310 covering them) extend at least as far as a maximum crimped diameter of the prosthetic heart valve 220. In embodiments, the retention bumpers may extend further radially outward than the prosthetic heart valve 220. Thus, the features of the balloon catheter 300 having the largest diameter are the enlarged balloon portions 302 formed by the balloon 310 wrapped over the retention bumpers 301 and not the prosthetic heart valve 220. When inserted into an introducer, the enlarged balloon portion 302 raised by the retention bumpers 301 is therefore the portion of the balloon catheter 300 that contacts the introducer wall, thereby preventing the wall of the introducer from contacting the prosthetic heart valve 220 and thereby reducing the amount of force acting on the prosthetic heart valve 220 from the inner walls of the introducer.

FIG. 2A illustrates the balloon catheter 300 in a neutral position with the prosthetic heart valve 220 crimped onto the balloon 310. FIG. 2B illustrates the balloon catheter 300 in a curved position. During valve delivery, the balloon catheter 300 is tracked at least partially inside an introducer sheath to a delivery site and must traverse the twists and turns of a patient's vascular anatomy. In particular, the aortic arch presents a tight turn for the balloon catheter 300 to traverse. When bending of the distal portion of the balloon catheter 300 is not required, as shown in FIG. 2A, the retention bumpers 301 operate to protect the prosthetic heart valve 220 from excessive contact with the introducer sheath and/or with the patient anatomy, as discussed above.

As illustrated in FIG. 2A, the retention bumpers 301 create a bulge in the balloon 310 where it is wrapped over the bumpers. This bulge, referred to herein as an enlarged balloon portion 302, is defined by the outer diameter or perimeter of the balloon 310 where it is wrapped over the retention bumpers 301. The enlarged balloon portion 302 has a diameter that is larger than that of the crimped prosthetic heart valve 220. When the balloon catheter 300 is advanced through the introducer sheath, the enlarged balloon portion 302 contacts the introducer sheath and acts to reduce, minimize, or eliminate, contact between the introducer sheath and the prosthetic heart valve. When the distal portion of the balloon catheter extends beyond the introducer sheath and into the patient vasculature, the enlarged balloon portion 302 aids in preventing contact between the prosthetic heart valve 220 and the patient vasculature. The cylinder volume between the enlarged balloon portions 302 wherein contact between the introducer sheath or patient vasculature and prosthetic heart valve 220 is reduced, minimized, or eliminated is referred to herein as the protection zone 385. The protection zone 385 is defined by a projected cylindrical volume between cross-sections of the enlarged balloon portions 302. The prosthetic heart valve 220 is radially aligned with the retention bumpers 301 when the entirety of a cross-section of the prosthetic heart valve 220 remains within the projection of the cross-section of the enlarged balloon portions 302. This arrangement is illustrated in FIGS. 2A and 3A. FIG. 3A is an illustration of a cross section of the balloon catheter 300 taken at line A-A. FIG. 3A illustrates the prosthetic heart valve 220 crimped to the balloon 310, which is wrapped over the inner shaft 330. The protection zone 385, representing the projection of the cross-section of the enlarged balloon portion 302, encompasses the prosthetic heart valve 220. When the balloon catheter 300 is advanced through an introducer sheath or extends out the distal end of an introducer sheath into the patient vasculature, the protection zone 385 represents the portion of the introducer sheath and/or patient vasculature over which contact with the prosthetic heart valve 220 is reduced, minimized, or eliminated due to the presence of the retention bumpers 301 and the enlarged balloon portions 302. When inside of the protection zone 385, the prosthetic heart valve 220 is less likely to contact the introducer sheath and/or patient vasculature.

When bending of the distal portion of the balloon catheter 300 is required, as shown in FIG. 3B, disparities between the stiffness of the balloon catheter 300 itself (i.e., the inner shaft 330 and balloon 310) and the stiffness of the prosthetic heart valve 220 can cause a radial misalignment of the prosthetic heart valve 220 with the retention bumpers 301.

As illustrated in FIGS. 2B and 3B, due to forces on the balloon catheter 300 caused by the curvature of the introducer sheath and/or patient vasculature, the balloon 310 and inner shaft 330 located underneath the prosthetic heart valve 220 bend. The prosthetic heart valve 220, however, is of a greater stiffness than these portions of the balloon 310 and inner shaft 330 and bends less than the catheter in this area. This disparity in curvature causes a distortion of the protection zone 385. The distortion of the protection zone 385 may cause portions of the prosthetic heart valve 220 to extend beyond a projection of the cross-section of the enlarged balloon portions 302, e.g., outside of the protection zone 385, where they may be more likely to contact the introducer sheath and/or patient vasculature. In particular, the distal crowns 227 and/or the proximal crowns 227 of the prosthetic heart valve 220 may protrude outside of the protection zone 385. The protrusion of the distal crowns 227 of the prosthetic heart valve 220 beyond the protection zone 385 is shown in FIGS. 2B and 3B. FIG. 3B illustrates a cross section of the flexed balloon catheter 300 taken at the line B-B. FIG. 3B illustrates the prosthetic heart valve 220 crimped to the balloon 310, which is wrapped over the inner shaft 330. The protection zone 385, representing the projection of the cross-section of the enlarged balloon portion 302 is distorted and does not fully encompass the prosthetic heart valve 220. The protrusions past the protection zone 385 of the retention bumpers 301 may result in the prosthetic heart valve catching on or even puncturing the introducer sheath. If the introducer sheath is punctured, the prosthetic heart valve may become stuck within the introducer and/or may cause damage to patient anatomy. In configurations where the distal portion of the balloon catheter extends beyond the introducer sheath, protruding portions of the prosthetic heart valve 220 may cause damage to the patient vasculature. FIGS. 2B and 3B illustrate protrusion of the distal crowns 227 beyond the protection zone 385. In further configurations, the proximal crowns 227 may extend beyond the protection zone 385.

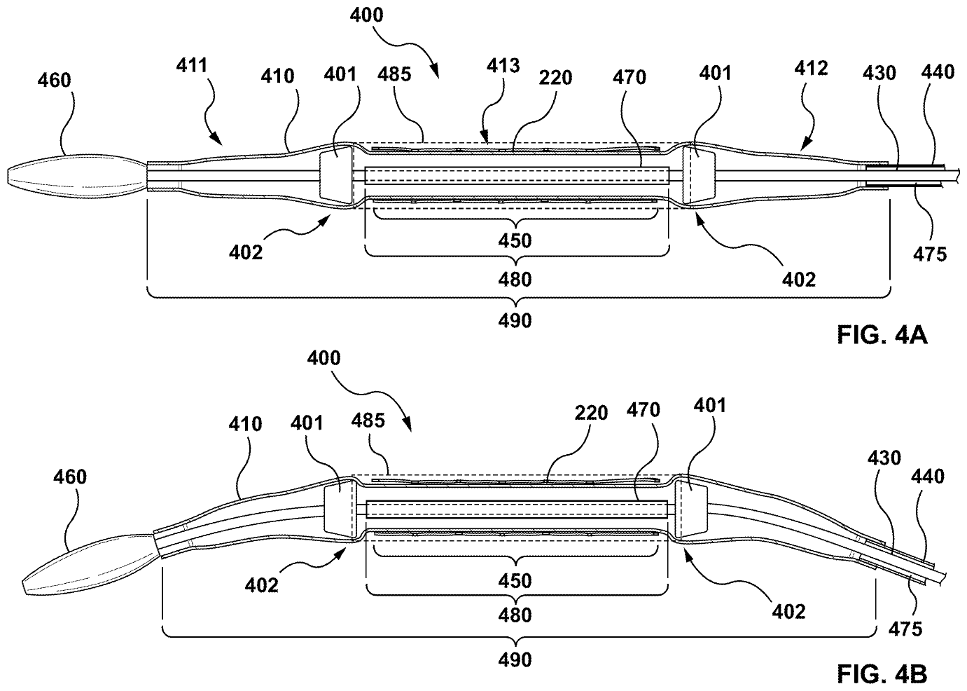

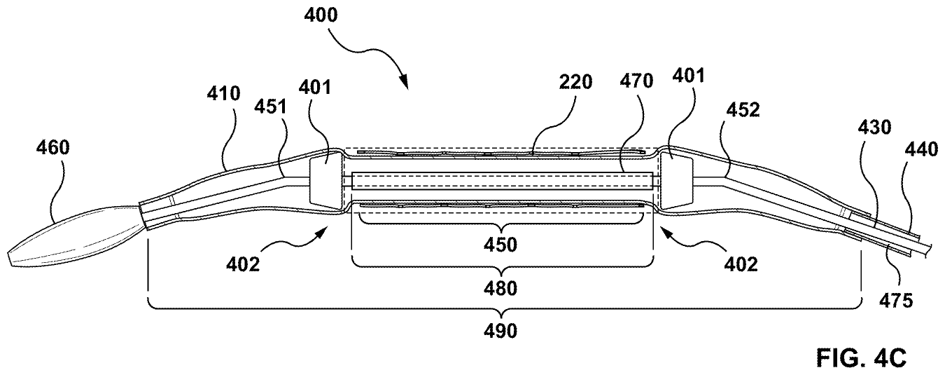

FIGS. 4A-4C illustrate cross sections of a balloon catheter 400 employing a reinforcing member 470 consistent with embodiments hereof. The reinforcing member 470 is configured to cause an increase in axial bending stiffness of the balloon catheter 400 in a reinforced portion 480 of the balloon catheter 400. The balloon catheter 400 includes an outer shaft 440, an inner shaft 430, a balloon 410, retention bumpers 401, and a distal tip 460. The balloon catheter 400 may have a prosthetic heart valve 220 crimped over the balloon 410. Inflation fluid is delivered to the balloon 410 via an inflation lumen 475 disposed between the outer shaft 440 and the inner shaft 430. The balloon 410 includes a distal portion 411, a proximal portion 412, and a central portion 413. The central portion 413 is the portion over which the prosthetic heart valve 220 is crimped. The balloon catheter 400 further includes proximal and distal retention bumpers 401.

The balloon 410 is wrapped over the retention bumpers 401 to create enlarged balloon portions 402 having an increased diameter with respect to the remainder of the balloon 410. Cross-section projections of the enlarged balloon portions 402 define a protection zone 485, similar to the protection zone 385. The portion of the balloon catheter 400 over which the balloon 410 is disposed defines a delivery portion 490 of the balloon catheter 400. The balloon catheter 400 further includes an operational portion 450 configured to receive the prosthetic heart valve 220 crimped thereto. The operational portion 450 is located between the proximal and distal retention bumpers 401 and corresponds to the central portion 413 of the balloon 410. The balloon catheter 400 also includes a reinforcing member 470. The reinforcing member 470 is disposed at least in the operational portion 450 of the balloon catheter 400. The reinforcing member 470 may be disposed strictly within the operational portion 450 (i.e., with a length smaller than or coinciding with the length of the prosthetic heart valve 220), strictly between the retention bumpers 401, and/or may extend past the retention bumpers 401.

The reinforcing member 470 is configured to increase the rigidity of the inner shaft 430 to provide a reinforced portion 480. The reinforced portion 480 may coincide with the operational portion 450 of the balloon catheter 400, may partially coincide with the operational portion 450, may coincide with a length between the retention bumpers 401, and/or may extend beyond either of these regions. As used herein, “coincide” refers to an approximately equal correspondence between the lengths and relative positions of the coinciding portions. As used herein, “partially coincide” refers to an overlap between partially coinciding portions where at least the lengths or relative positions of the partially coinciding portions are not approximately equal.

The reinforcing member 470 is a tube disposed over the inner shaft 430 over the length of the reinforced portion 480. In embodiments, the reinforcing member 470 is a hypo tube (e.g., stainless steel, MP35N, elgiloy, and L605), a plastic tube, a polymer tube (e.g., PEEK, pebax, polyester, fluorocarbon, polyethylene, polyurethanes, nylon, acetal, etc.), and/or may be a tube of any suitable material. In further embodiments, the reinforcing member 470 may be a composite structure such as a metal braid with polymer jacket and liners, metal coils with polymer jacket and liners, polymer braids such as PEEK or Kevlar and polymer jackets and liners and polymer coils such as PEEK or Kevlar and polymer jackets and liners. The reinforcing member 470 may be attached or secured to the inner shaft 430 via any suitable means, including swaging, bonding, overmolding, adhesives, etc. In embodiments, the reinforcing member 470 is configured with a low profile and lays against the inner shaft 430 with no gap therebetween.

In further embodiments, a gap between the reinforcing member 470 and the inner shaft 430 is permitted and may be achieved or maintained via spacers, for example. For example, the reinforcing member 470 may be spaced away from the inner shaft 430 and may permit inflation fluid flow through the annular space between an outer surface of the inner shaft 430 and an inner surface of the reinforcing member 470.

The reinforcing member 470 increases the rigidity of the balloon catheter 400 over the reinforced portion 480. The reinforced portion 480 coincides with the reinforcing member 470. It is not required that the reinforcing member 470, by itself, have a rigidity greater than the inner shaft 430. Combining the reinforcing member 470 with the inner shaft 430 results in a rigidity greater than the inner shaft 430 alone. Thus, the reinforcing member 470 may have a rigidity greater than the inner shaft 430 or may have a rigidity less than the inner shaft 430. The reinforcing member 470 increases the rigidity of the balloon catheter 400 in the reinforced portion 480 such that it is greater than the rigidity of the balloon catheter 400 in the remaining portions of the delivery portion 490.

In embodiments, the reinforcing member 470 may be configured so as to increase the rigidity of the reinforced portion 480 such that the reinforced portion 480 has a rigidity substantially equal to the rigidity of the prosthetic heart valve 220. In embodiments, the reinforcing member 470 may be configured to increase the rigidity of the reinforced portion 480 such that the reinforced portion 480 has a rigidity greater than 90%, greater than 80%, greater than 70%, greater than 60%, greater than 50%, or greater than 40% of the rigidity of the prosthetic heart valve 220. In embodiments, the rigidity of the reinforced portion 480 may be greater than the rigidity of the prosthetic heart valve 220. In embodiments, the rigidity of the reinforced portion 480 may vary over the length of the reinforced portion 480. For example, the reinforcing member 470 may be provided to achieve a reinforced portion 480 rigidity of 50% of the prosthetic heart valve at the respective proximal and distal ends of the reinforced portion 480 with a smooth or abrupt transition to a rigidity of 80% of the prosthetic heart valve in the middle. In embodiments, the rigidity of the reinforced portion 480 may smoothly transition from a maximum rigidity of the reinforced portion 480 at a position close to the ends of the prosthetic heart valve 220 (e.g., between 1-3 mm from the end) to a rigidity equal to the remainder of the inner shaft 430 at a position several mms outside the operational portion 450 and away from the ends of the prosthetic heart valve 220 (e.g., between 3-10 mms distal and/or proximal of the respective distal and proximal ends of the prosthetic heart valve 220). The specific percentages provided are by way of example only, and the varying rigidity over the length of the reinforcing portion may be selected with any suitable stiffness profile to prevent protrusion of the prosthetic heart valve 220, as described below.

The increased rigidity of the inner shaft 430 over the reinforced portion 480 alters the manner in which the operational portion 450 of the balloon catheter 400 curves when the balloon catheter 400 is tracked through a patient's vasculature. FIG. 4A illustrates the balloon catheter 400 in a neutral position while FIG. 4B illustrates the balloon catheter 400 in a curved position. When subject to curving, the increased stiffness of the balloon catheter 400 over the reinforced portion 480 causes the balloon catheter 400 to show greater curvature at locations outside the reinforced portion 480. The increased stiffness of the balloon catheter 400 over the reinforced portion 480 causes the balloon catheter 400 to remain straighter over the reinforced portion 480 than over the remaining portions of the delivery portion 490. Locations outside of the reinforced portion 480 include remaining locations within the delivery portion 490. In some embodiments, the balloon catheter 400 may remain substantially straight over the length of the reinforced portion 480. In some embodiments, the balloon catheter 400 may bend and show lesser curvature over the reinforced portion 480 and greater curvature in the delivery portion 490 outside of the reinforced portion 480. In some embodiments, the balloon catheter 400 may have a “flex point” outside of the reinforced portion 480 where the curvature of the balloon catheter 400 changes abruptly. In embodiments, as illustrated in FIG. 4C, the balloon catheter 400 may have a first flex point 451 outside of one end of the reinforced portion 480 and a second flex point 452 outside of a second, opposing, end of the reinforced portion 480.

Due to the increased rigidity of the reinforced portion 480 (which at least partially coincides with the operational portion 450) of the balloon catheter 400, distortion of the protection zone 485 is reduced or eliminated. The prosthetic heart valve 220 remains in the protection zone 485, i.e. in radial alignment with the retention bumpers 401 and therefore does not radially project or protrude from the cross-section projection of the enlarged balloon portions 402. The prosthetic heart valve 220 does not radially protrude or extend past the diameter of the retention bumpers 401. This alignment preservation is illustrated in FIG. 4B. Accordingly, the above-discussed problems, including introducer sheath damage or puncture and anatomy damage, can be avoided.

As discussed above, the reinforced portion 480 may partially or completely coincide with the operational portion 450. The level of coincidence between the reinforced portion 480 and the operational portion 450 is selected to sufficiently increase the rigidity of the balloon catheter 400 to preserve radial alignment of the prosthetic heart valve 220 with the retention bumpers 401. In embodiments, this may be achieved by a reinforcing member 470 (and reinforcing portion 480) shorter than, equal in length to, or longer than the operational portion 450. Additional considerations in selecting the length of the reinforcing member 470 (and reinforcing portion 480) may include manufacturability, cost, and overall operational characteristics of the balloon catheter 400. For example, making the reinforcing member 470 too long may preserve radial alignment of the prosthetic heart valve 220 with the retention bumpers 401 while sacrificing overall catheter maneuverability.

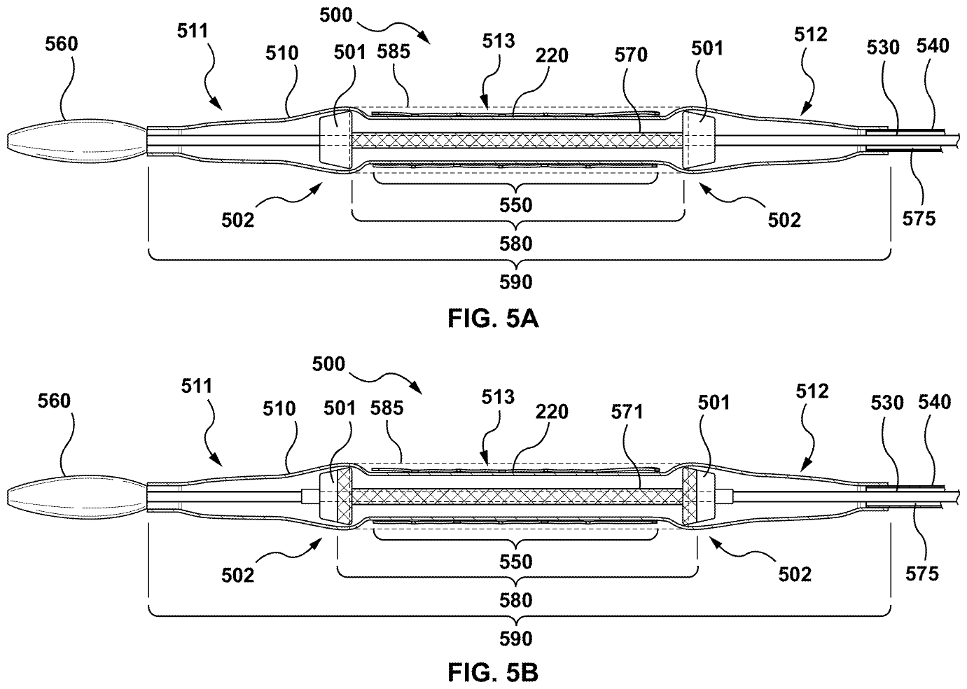

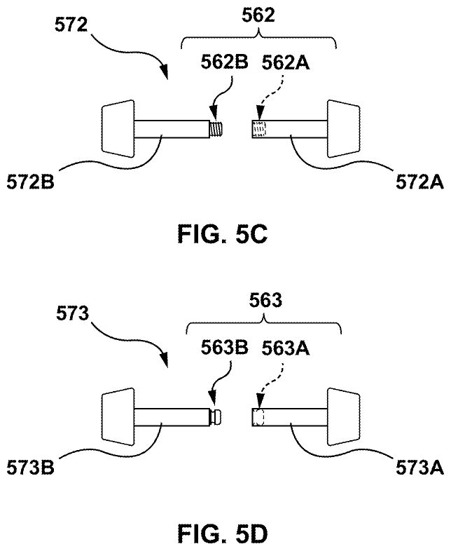

FIGS. 5-12 illustrate further embodiments of reinforcing members and retention bumpers applied to balloon catheters. The balloon catheters, reinforcing members, and retention bumpers described with respect to FIGS. 5-12 include all of the functionality and variations described above with respect to the reinforcing member 470 and the balloon catheter 400, including variations in material, size, stiffness profile, and other features.

In embodiments, reinforcing members are configured to span a gap between the prosthetic heart valve and the retention bumpers. Both the prosthetic heart valve and the retention bumpers provide stiffness to the balloon catheter. However, absent further reinforcing features, the gap between the prosthetic heart valve and the retention bumpers creates an abrupt drop in the stiffness of the balloon catheter in the gap. Accordingly, the stiffness profile of the balloon catheter over the delivery portion is not smooth. When subject to bending stresses over the length of the balloon catheter, these areas of reduced stiffness may undergo significantly more strain than the stiffer surrounding areas. This additional strain may cause kinks at these reduced stiffness gaps. Accordingly, embodiments described herein may be configured to reinforce the reduced stiffness gaps between the retention bumpers and the prosthetic heart valves. Such reinforcement may be provided by specific reinforcement members and/or by retention bumpers configured with features for reinforcing the gap between the prosthetic heart valve and the retention bumpers.

FIG. 5A illustrates a reinforcing member embodiment wherein the reinforcing member is formed integrally with the retention bumpers. FIG. 5A illustrates a balloon catheter 500 including an outer shaft 540, an inner shaft 530, a balloon 510, and a distal tip 560. A prosthetic heart valve 220 may be crimped to the balloon 510 of the balloon catheter 500. Inflation fluid is delivered to the balloon 510 via an inflation lumen 575 disposed between the outer shaft 540 and the inner shaft 530. The balloon 510 includes a distal portion 511, a proximal portion 512, and a central portion 513. The central portion 513 is the portion over which the prosthetic heart valve 220 is crimped.

The portion of the balloon catheter 500 over which the balloon 510 is disposed defines a delivery portion 590 of the balloon catheter 400. The balloon catheter 500 further includes an operational portion 550 configured to receive the prosthetic heart valve 220 crimped thereto. The operational portion 550 is located between the proximal and distal retention bumpers 501 and corresponds to the central portion 513 of the balloon 510.

In the balloon catheter 500, the reinforcing member 570 includes the proximal and distal retention bumpers 501 formed integrally therewith. A proximal reinforcing member portion is integral with the proximal retention bumper 501 and a distal reinforcing member portion is integral with the distal retention bumper 501. The balloon 510 is wrapped over the retention bumpers 501 to create enlarged balloon portions 502 having an increased diameter with respect to the remainder of the balloon 510. Cross-section projections of the enlarged balloon portions 502 define a protection zone 585, similar to protection zones 385 and 485. The proximal and distal retention bumpers 501 establish the protection zone 585. The reinforcing member 570 defines the reinforced portion 580, over which the rigidity of the balloon catheter 500 is increased relative to portions outside of the reinforced portion 580. The reinforcing member 570 may be injection molded and then bonded or adhered to the inner shaft 530. The reinforcing member 570 may also be formed via overmolding. The reinforcing member 570 may improve the manufacturability of the balloon catheter 500 by reducing the total number of parts and/or by ensuring proper distancing between the retention bumpers 501 during the assembly process.

FIG. 5B illustrates a reinforcing member embodiment wherein the reinforcing member is formed integrally with the retention bumpers and has a variable stiffness. The reinforcing member 571 includes the proximal and distal retention bumpers 501 formed integrally therewith. The proximal and distal retention bumpers establish the protection zone 585. The reinforcing member 571 extends proximally and distally of the integrally formed retention bumpers 501. The reinforcing member 571 includes a transition from a more rigid central portion to more flexible proximal and distal portions. The transition from rigid to flexible may occur at the location of the integral retention bumpers 501, as illustrated in FIG. 5B. In alternative embodiments, the transition may occur inside of or outside of the length spanned by the integral retention bumpers 501. The reinforcing member 571 defines the reinforced portion 580, over which the rigidity of the balloon catheter 500 is increased relative to portions outside of the reinforced portion 580. The reinforcing member 571 may be injection molded and then bonded or adhered to the inner shaft 530, e.g., via welding, adhesive, reflowing, etc. The reinforcing member 571 may also be formed via overmolding of the inner shaft 530. In further embodiments, the reinforcing member 571 may take the place of a portion of the inner shaft 530 and may be attached, for example by butt-welding, to the proximal and distal portions of the inner shaft 530. The reinforcing member 571 may improve the manufacturability of the balloon catheter 500 by reducing the total number of parts and/or by ensuring proper distancing between the retention bumpers 501 during the assembly process. The variable stiffness of the reinforcing member 571 may be achieved through various means, for example through different material thicknesses, differing material selection, inclusion of a braided jacket in the rigid portion, and any other suitable means. The variable stiffness of the reinforcing member 571 may also be achieved by altering the pic rate of a braided shaft present through portions of the inner shaft 530 extending past the operational portion 550. For example, a braided shaft extending the length of the inner shaft 530 may have an altered pic rate at the reinforced portion 580 to generate reinforcing member 570.

FIGS. 5C and 5D illustrate two part reinforcing members including retention bumpers. FIG. 5C illustrates a reinforcing member 572 formed from a first portion 572A and a second portion 572B. Each of the portions of the reinforcing member 572 includes a retention bumper 501 formed integrally therewith. The first portion 572A and the second portion 572B are configured to interlock with each other to form the reinforcing member 572. To maintain strength at the interlocking portion, the reinforcing member 572 has overlapping solid features at the joint. For example, as illustrated in FIG. 5C, interlock may be achieved via a screw fit 562. For example, the screw fit 562 may be created by interior threads 562A on the first portion 572A configured to interlock with exterior threads 562B of the second portion 572B. In embodiments, the location of the interior threads 562A and exterior threads 562B may be interchanged. In further embodiments, other means of interlock having overlapped solid features may be employed. For example, FIG. 5D illustrates a reinforcing member 573 formed from a first portion 573A and a second portion 573B that employs a snap fitting 563. For example, the snap fitting 563 may be created by a contoured recess 563A on the first portion 573A configured to interlock with a ridged projection 563B on the second portion 573B. In embodiments, the location of the contoured recess 563A and the ridged projection 563B may be interchanged. Further embodiments may include additional overlapping features, such as a rotational interlock. The reinforcing members 572 or 573 define a reinforced portion (not shown), over which the rigidity of the balloon catheter 500 is increased relative to portions outside of the reinforced portion. The first portion 572A and the second portion 572B of the reinforcing member 572 may be injection molded and then bonded or adhered to the inner shaft 530 after interlock.

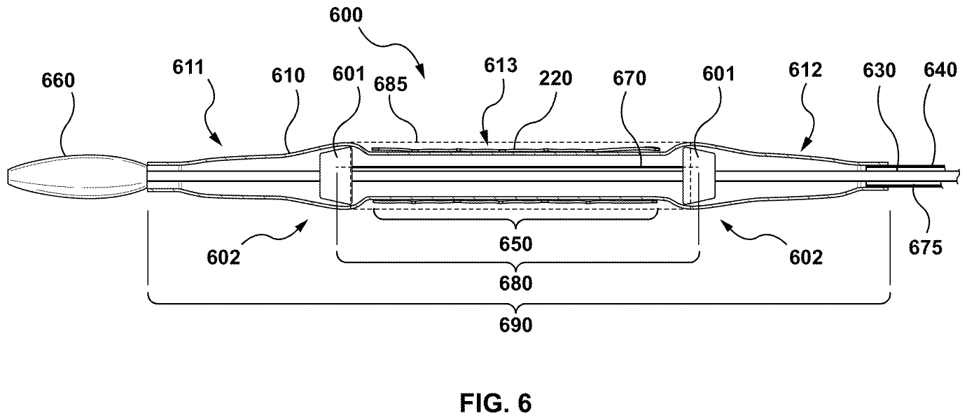

FIG. 6 illustrates a reinforcing member embodiment wherein the reinforcing member is provided as a wire. FIG. 6 illustrates a balloon catheter 600. The balloon catheter 600 includes an outer shaft 640, an inner shaft 630, a balloon 610, a distal tip 660, and retention bumpers 601. A prosthetic heart valve 220 may be crimped to the balloon 610. The retention bumpers 601 establish a protection zone 685. Inflation fluid is delivered to the balloon 610 via an inflation lumen 675 disposed between the outer shaft 640 and the inner shaft 630. The balloon 610 includes a distal portion 611, a proximal portion 612, and a central portion 613. The central portion 613 is the portion over which the prosthetic heart valve 220 is crimped.

The balloon 610 is wrapped over the retention bumpers 601 to create enlarged balloon portions 602 having an increased diameter with respect to the remainder of the balloon 610. Cross-section projections of the enlarged balloon portions 602 define a protection zone 685, similar to protection zones 385, 485, and 585. The portion of the balloon catheter 600 over which the balloon 610 is disposed defines a delivery portion 690 of the balloon catheter 600. The balloon catheter 600 further includes an operational portion 650 configured to receive the prosthetic heart valve 220 crimped thereto. The operational portion 650 is located between the proximal and distal retention bumpers 601 and corresponds to the central portion 613 of the balloon 610.

In the balloon catheter 600, the reinforcing member 670 is a wire, rod, or tube secured to the exterior of the inner shaft 630. The reinforcing member 670 extends along the exterior of the inner shaft 630 and has a longitudinal axis outside of the diameter of the inner shaft 630 and does not fully encompass the inner shaft 630. The reinforcing member 670 may have any suitable cross-sectional shape, e.g., circular, oval, rectangular, square, etc. In embodiments, the reinforcing member 670 may be curved so as to conform to a portion of the circumference of the inner shaft 630. The reinforcing member 670 defines the reinforced portion 680, over which the rigidity of the balloon catheter 600 is increased relative to portions outside of the reinforced portion 680. The reinforcing member 670 may bonded, adhered, or clamped to the inner shaft 630. In some embodiments, the reinforcing member 670 may extend into or be secured to the retention bumpers 601. The reinforced portion 680 is at least partially coincidental with the operational portion 650, and may be shorter, longer or the same length as the operational portion 650.

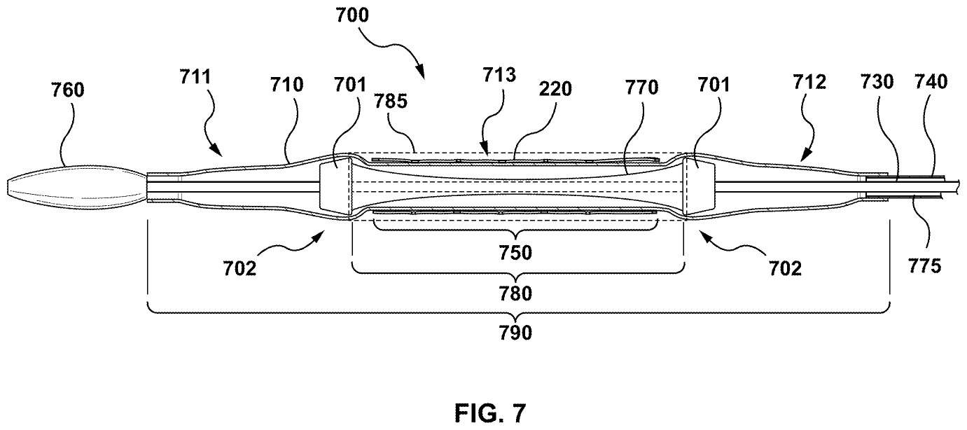

FIG. 7 illustrates a reinforcing member embodiment wherein the reinforcing member is provided as a collar providing variable stiffness throughout the reinforced portion. FIG. 7 illustrates a balloon catheter 700 including an outer shaft 740, an inner shaft 730, a balloon 710, a distal tip 760, and retention bumpers 701. The prosthetic heart valve 220 is crimped to the balloon 710. Inflation fluid is delivered to the balloon 710 via an inflation lumen 775 disposed between the outer shaft 740 and the inner shaft 730. The balloon 710 includes a distal portion 711, a proximal portion 712, and a central portion 713. The central portion 713 is the portion over which the prosthetic heart valve 220 is crimped.

The balloon 710 is wrapped over the retention bumpers 701 to create enlarged balloon portions 702 having an increased diameter with respect to the remainder of the balloon 710. Cross-section projections of the enlarged balloon portions 702 define a protection zone 785, similar to protection zones 385, 485, 585, and 685. The portion of the balloon catheter 700 over which the balloon 710 is disposed defines a delivery portion 790 of the balloon catheter 700. The balloon catheter 700 further includes an operational portion 750 configured to receive the prosthetic heart valve 220 crimped thereto. The operational portion 750 is located between the proximal and distal retention bumpers 701 and corresponds to the central portion 713 of the balloon 710. In the balloon catheter 700, the reinforcing member 770 is a structure having a varying annular cross-section disposed between the retention bumpers 701. The reinforcing member 770 is disposed over the inner shaft 730 and may be coaxial with the inner shaft 730. The cross-section of the reinforcing member 770 is annular, having an interior hollow to accommodate the inner shaft 730. The inner diameter of the reinforcing member 770 is constant over its length and the outer-diameter is variable, creating a varying annular cross-section. The retention bumpers 701 establish the protection zone 785. The reinforcing member 770 may be formed separately or integrally with the retention bumpers 701. The reinforcing member 770 defines the reinforced portion 780, over which the rigidity of the balloon catheter 700 is increased relative to portions outside of the reinforced portion 780. The reinforcing member 770 may be injection molded and then bonded or adhered to the inner shaft 730. The reinforcing member 770 may also be formed via overmolding. The reinforcing member may be formed from any suitable material, including polymers such as Pebax, nylon, acetal, high density poly-ethylene, and others. The reinforcing member 770 may have a thickness profile that varies according to the varying annular cross-section to tailor the stiffness throughout the reinforced portion 780. The thickness profile of the reinforcing member 770 is selected to provide the reinforced portion 780 with a desired variable stiffness profile and thus provide the balloon catheter 700 with a selected bending curvature profile.

The illustrated embodiment of the reinforcing member 770 has an end-weighted thickness profile that reaches a maximum at the distal and proximal ends of the reinforcing member 770 and a minimum at or near the middle of the reinforcing member 770. This end-weighted thickness profile leads to a greater stiffness at the distal and proximal ends of the reinforcing member 770. In further embodiments, alternate thickness profiles are consistent with this disclosure. For example, the reinforcing member may be formed with a center-weighted thickness profile, wherein the thickness or diameter of the reinforcing member 770 (and thus the stiffness) is greatest at or near the center of the reinforcing member 770. Other suitable thickness profiles of the reinforcing member 770 may also be employed.

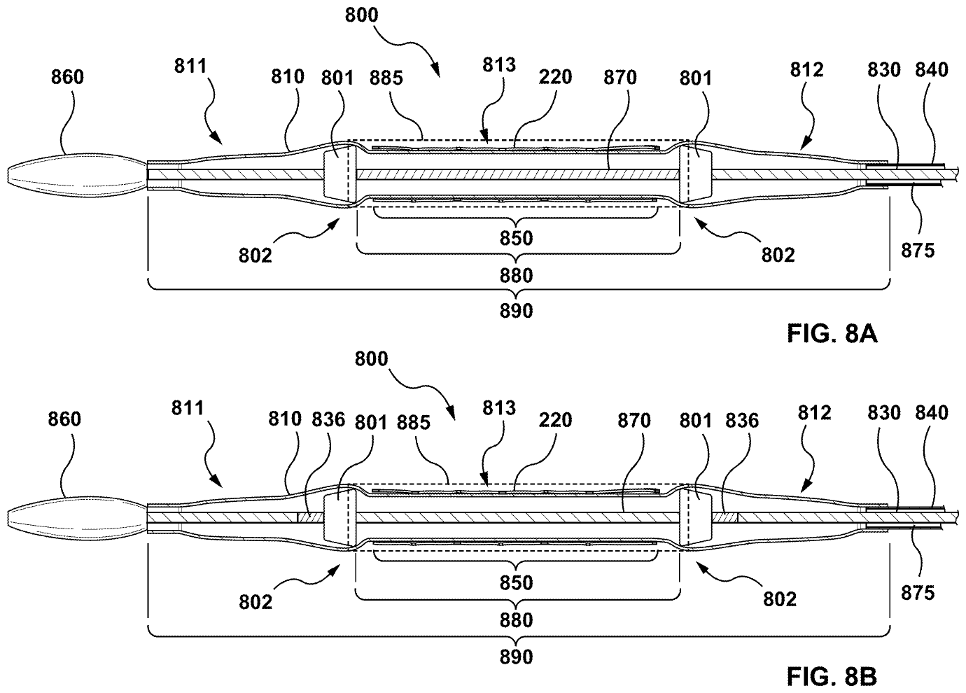

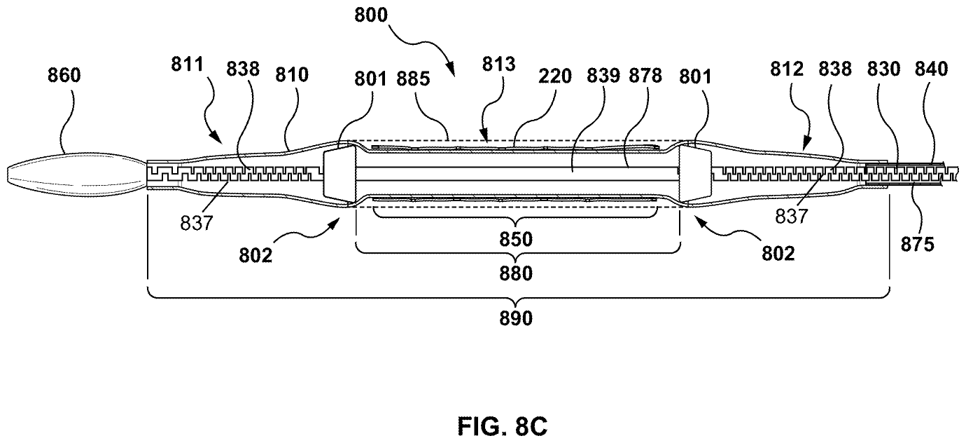

FIGS. 8A-8C illustrate a reinforcing member incorporated into an inner shaft. FIGS. 8A-8C illustrate a balloon catheter 800 including an outer shaft 840, an inner shaft 830, a balloon 810, a distal tip 860, and retention bumpers 801. The prosthetic heart valve 220 is crimped to the balloon 810. The balloon catheter 800 also includes retention bumpers 802 that establish a protection zone 885. Inflation fluid is delivered to the balloon 810 via an inflation lumen 875 disposed between the outer shaft 840 and the inner shaft 830. The balloon 810 includes a distal portion 811, a proximal portion 812, and a central portion 813. The central portion 813 is the portion over which the prosthetic heart valve 220 is crimped.

The balloon 810 is wrapped over the retention bumpers 801 to create enlarged balloon portions 802 having an increased diameter with respect to the remainder of the balloon 810. Cross-section projections of the enlarged balloon portions 802 define a protection zone 885, similar to protection zones 385, 485, 585, 685, and 785. The portion of the balloon catheter 800 over which the balloon 810 is disposed defines a delivery portion 890 of the balloon catheter 800. The balloon catheter 800 further includes an operational portion 850 configured to receive the prosthetic heart valve 220 crimped thereto. The operational portion 850 is located between the proximal and distal retention bumpers 801 and corresponds to the central portion 813 of the balloon 810. In the balloon catheter 800, the reinforcing member 870 is incorporated into the inner shaft 830 as a portion of the inner shaft 830. The reinforcing member 870 defines the reinforced portion 880, over which the rigidity of the balloon catheter 800 is increased relative to portions outside of the reinforced portion 880.

In FIG. 8A, the reinforcing member 870 is a portion of the inner shaft 830 with an increased rigidity compared to the remainder of the inner shaft 830. The reinforcing member 870 may be a stiffened portion of the inner shaft 830. The reinforcing member 870 may be formed from a material more rigid than the remainder of the inner shaft 830. The reinforcing member 870 may be formed by incorporating one or more wires or braids inset into the walls of the inner shaft 830. The reinforcing member 870 may additionally be formed from a member having a thicker wall than the remainder of the inner shaft 830. The reinforcing member 870 may be formed in any suitable fashion. In an embodiment, the reinforcing member 870 may be formed during manufacture of the inner shaft 830 by altering the composition (e.g., different material, inserted wires or braids, etc.) of the reinforcing member 870 portion of the inner shaft 830 during manufacture of the inner shaft 830. In further embodiments, the reinforcing member 870 may be formed separately and then laser welded (e.g., butt welded) with the portions of the inner shaft 830 that are proximal and distal of the reinforcing member 870.

In embodiments, the reinforcing member 870 may be a portion of the inner shaft 830 having increased rigidity as compared to neighboring portions of the inner shaft 830. Thus, the reinforcing member 870 may be formed by reducing rigidity of the inner shaft 830 in neighboring portions of the inner shaft 830 outside of the reinforced portion 880. FIG. 8B illustrates a reinforcing member 870 generated by reductions in inner shaft rigidity outside of the reinforced portion 880. Reduced stiffness portions 836, at either side of the reinforcing member 870 on the inner shaft 830, have a reduced rigidity. The rigidity in these portions may be reduced, for example, by laser cutting material away from the inner shaft 830 in the reduced stiffness portions 836. Laser cutting material may include adding holes or skives and/or reducing a diameter of the inner shaft 830 in the reduced stiffness portions 836. Reducing the rigidity of the inner shaft 830 outside of the reinforced portion 880 creates a greater likelihood of flexing or increased curvature outside of the reinforced portion 880 or the operational portion 850, thereby maintaining radial alignment between the retention bumpers and the prosthetic heart valve 220. The reinforcing member 870 may have a rigidity substantially the same as the rigidity of the remainder of the inner shaft 830 (excluding the reduced stiffness portion 836).

FIG. 8C illustrates a reinforcing member attached to the inner shaft according to embodiments hereof. The reinforcing member 878 may be incorporated into the balloon catheter 800 in place of the reinforcing member 870 of FIGS. 8A-8B. The reinforcing member 878 is a hypotube having reduced stiffness portions 838 at either end and a central portion 839 between the reduced stiffness portions 838. The central portion 839 of the reinforcing member 878 generates the reinforced portion 880 of the balloon catheter 800. The reinforced portion 880 extends at least to the retention bumpers 801, thereby reinforcing the gap between the prosthetic heart valve 220 and the retention bumpers 801.

The reduced stiffness portions 838, at either side of the central portion, have a reduced bending stiffness. The reduced stiffness is generated by material reductions 837 in the hypotube. The material reductions 837 may be holes, skives, slots, perforations, or features that remove material from the hypotube. The material reductions 837 may be created via any suitable manufacturing process, including laser cutting. The central portion 839 of the reinforcing member spans the reinforced portion 880 of the balloon catheter 800, between the retention bumpers 801. The reduced stiffness portions 838 begin within the retention bumpers 801 and extend proximally of the proximal retention bumper 801 and distally of the distal retention bumper 801. The reinforcing member 878 extends proximally and distally of the retention bumpers 801 at least as far as the proximal portion 812 and the distal portion 811 of the balloon 810. Thus, the reinforcing member 878 extends at least as far as the limits of the delivery portion 890. In embodiments, the reinforcing member 878 may extend beyond the limits of the delivery portion 890.

The central portion 839 of the reinforcing member 878 creates the reinforced portion 880 of the balloon catheter 800, which reduces, minimizes, or eliminates kinking over the reinforced portion 880. As discussed above, the reinforcing member 878 is also configured to reinforce the gap between the prosthetic heart valve 220 and the retention bumpers 801. Moderate bending outside of the reinforced portion 880 is further facilitated by the inclusion of the reduced stiffness portions 838. In embodiments, the material reductions 837 may be selectively generated to provide an optimal bending profile according to expected deployment conditions for the balloon catheter 800.

A bending profile may include varying bending stiffness over the length of the reinforcing member 878. In embodiments, bending stiffness may be varied abruptly, wherein the central portion 839 of the reinforcing member 878 is stiffer than the reduced stiffness portions 838. In further embodiments, bending stiffness may be varied gradually or continuously. For example, the material reductions 837 in the reduced stiffness portions 838 may be configured to provide multiple different stiffnesses in the reduced stiffness portions 838. In embodiments, multiple different stiffnesses may include different stiffnesses portions with abrupt changes therebetween, created, for example, by altering the pattern of the material reductions 837. In further embodiments, multiple different stiffnesses in the reduced stiffness portions 838 may include a continuously changing stiffness created, for example, by a continuous change in the pattern of the material reductions 837. For example, the reinforcing member 878 may be spiral cut with the distance between spirals changing gradually to continuously alter the stiffness. In another example, the distance between material reductions 837 may vary between each slit or skive such that the stiffness changes over the length of the portion having the material reductions 837. In embodiments, the varying profile of the bending stiffness in the reduced stiffness portion 838 may operate to provide strain relief and/or resistance to kinking and buckling between the outer shaft 840 and the proximally located retention bumper 802. The reduced stiffness portion may be configured so as to ease or mitigate an otherwise abrupt transition in stiffness at the point where the outer shaft 840 terminates.

In further embodiments, stiffness in the central portion 839 may also vary across the length of the central portion 839. The central portion 839 may include material reductions 837 to modify the stiffness in the central portion 839. Accordingly, the bending stiffness of the reinforcing member 878 may vary (smoothly or discretely) across the entirety of the reinforcing member 878, from the distal reduced stiffness portion 838, through the central portion 839, and through the proximal reduced stiffness portion 838.

In embodiments, the reinforcing member 878 may be disposed over the inner shaft 830. The reinforcing member 878 may be secured to the inner shaft 830 via one of several techniques. In embodiments, a distal end of the reinforcing member 878 is secured by the distal tip 860 when the distal tip is overmolded to the inner shaft 830. In embodiments, the inner shaft 830 may be reflowed over or around the reinforcing member 878, for example, through the material reductions 837 in the reinforcing member 878 so as to bond with and secure the reinforcing member 878. In embodiments, the retention bumpers 801 may secure the reinforcing member when they are overmolded to the inner shaft 830. The material reductions 837 provide gaps through which the material of the retention bumpers 801 may flow during overmolding. Thus, the material of the retention bumpers 801 extends through the material reductions 837 to bond to the inner shaft 830, thus securing the reinforcing member 878.

In further embodiments, the reinforcing member 878 may be configured as a part of the inner shaft 830, i.e., replacing a portion of the inner shaft 830 and being attached in line thereto. Accordingly, the reinforcing member 878 may constitute a distal portion of the inner shaft 830 and may be bonded to the remaining proximal portion of the inner shaft 830, e.g., via butt welding or other suitable technique.

In further embodiments, the reinforcing member 878 may include different materials and/or construction. For example, various polymers, plastics, and elastomers may be used to provide the reinforcing member 878. Stiffness of the reinforcing member 878 may be varied by altering the thickness and/or altering the material composition of the reinforcing member 878 over its length.

In further embodiments, the reinforcing members discussed above with respect to FIGS. 8A-8C may be used or employed without retention bumpers. The selective stiffening provided by the reinforcing members may serve to reduce or prevent crown protrusion in a balloon delivery catheter through the control of bending that is provided by the reinforcing members and without the added protection of the retention bumpers.

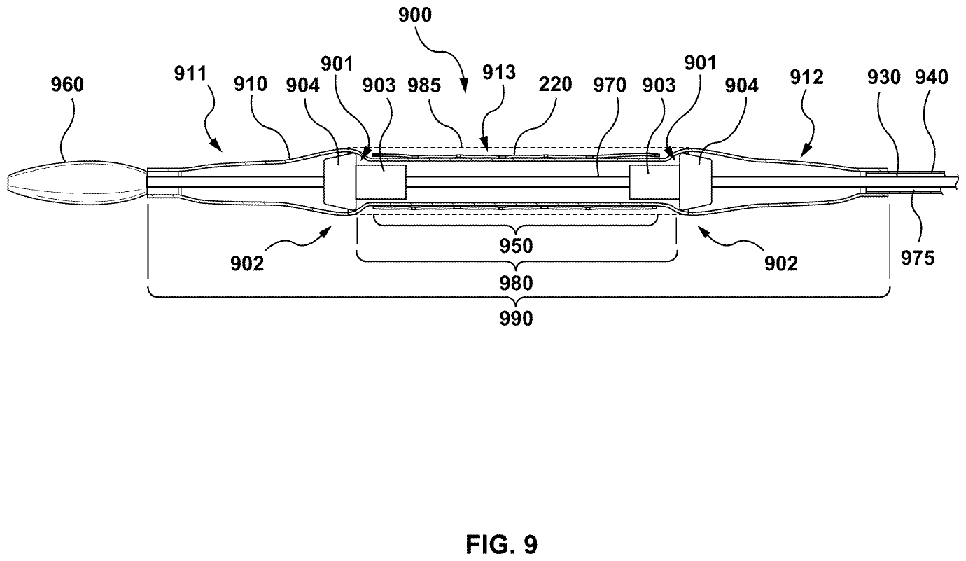

FIG. 9 illustrates extended retention bumpers configured to reduce or prevent crown protrusion in a balloon delivery catheter. FIG. 9 illustrates a balloon catheter 900 including an outer shaft 940, an inner shaft 930, a balloon 910, and a distal tip 960. A prosthetic heart valve 220 may be crimped on to the balloon 910 of the balloon catheter 900. Inflation fluid is delivered to the balloon 910 via an inflation lumen 975 disposed between the outer shaft 940 and the inner shaft 930. The balloon 910 includes a distal portion 911, a proximal portion 912, and a central portion 913. The central portion 913 is the portion over which the prosthetic heart valve 220 is crimped.

The portion of the balloon catheter 900 over which the balloon 910 is disposed defines a delivery portion 990 of the balloon catheter 900. The balloon catheter 900 further includes an operational portion 950 configured to receive the prosthetic heart valve 220 crimped thereon. The operational portion 950 is located between proximal and distal retention bumpers 901 and corresponds to the central portion 913 of the balloon 910.

In the balloon catheter 900, the retention bumpers 901 each include a reinforcement portion 903 and a migration prevention portion 904. The migration prevention portions 904 have a larger diameter than the reinforcement portions 903 and are configured similarly to other retention bumpers discussed herein. The diameter of the migration prevention portions 904 is large enough to create enlarged balloon portions 902 and establish a protection zone 985, similar to those discussed with respect to other embodiments herein. The reinforcement portions 903 are cylindrical projections extending from the migration prevention portions 904 with an axial hollow to accommodate the inner shaft 930. The diameter of the reinforcement portions 903 is small enough so as not to interfere with the crimping of the prosthetic heart valve 220.

The reinforcement portion 903 of each retention bumper 901 extends into the operational portion 950. The reinforcement portion 903 of the distal retention bumper 901 extends proximally of the migration prevention portion 904 of the distal retention bumper 901. The reinforcement portion 903 of the proximal retention bumper 901 extends distally of the migration prevention portion 904 of the proximal retention bumper 901. The balloon 910 is wrapped over the migration prevention portions 904 of the retention bumpers 901 to create enlarged balloon portions 902 having an increased diameter with respect to the remainder of the balloon 910 when the balloon is uninflated. Cross-section projections of the enlarged balloon portions 902 define a protection zone 985, similar to protection zones discussed above. The proximal and distal retention bumpers 901 establish the protection zone 985. The reinforcement portions 903 of the retention bumpers 901 define the outside axial ends of a reinforced portion 980 of the balloon catheter 900.

The reinforcement portions 903 of the retention bumpers 901 provide an increased stiffness or rigidity to the axial ends of the reinforced portion 980, thereby providing reinforcement to the gap between the prosthetic heart valve 220 and the migration prevention portions 904 of the retention bumpers 901. The reinforcement portions 903 provide an increased stiffness to the balloon catheter 900 over an extended length as compared to the retention bumpers 901 alone. The longer length of increased stiffness permits a gradual bending within the longer length and thereby reduces the likelihood of catheter kinking outside of the reinforced portion 980. The reinforcement portions 903 may extend axially from the migration prevention portions 904 by a distance in the range of 1-8 mm or the range of 2-5 mm.

The retention bumpers 901 may be injection molded and then bonded or adhered to the inner shaft 930. The retention bumpers 901 may also be formed via overmolding. The retention bumpers 901 may improve the manufacturability of the balloon catheter 900 by reducing the total number of parts and/or by ensuring proper distancing between the retention bumpers 901 during the assembly process when overmolded. The retention bumpers 901 are manufactured of stiff polymers, such as Pebax 55, Grilamid, or other polymers with similar stiffnesses. In embodiments, the reinforcement portions 903 of the retention bumpers 901 may be patterned to create a variable stiffness over their length.

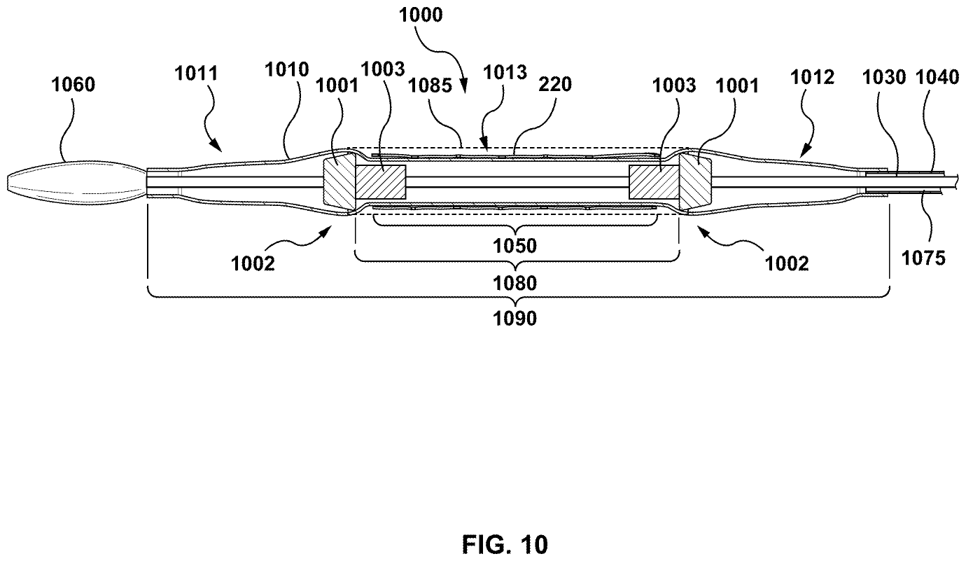

FIG. 10 illustrates reinforcement members configured to prevent or reduce crown protrusion in a balloon delivery catheter. FIG. 10 illustrates a balloon catheter 1000 including an outer shaft 1040, an inner shaft 1030, a balloon 1010, and a distal tip 1060. A prosthetic heart valve 220 may be crimped on to the balloon 1010 of the balloon catheter 1000. Inflation fluid is delivered to the balloon 1010 via an inflation lumen 1075 disposed between the outer shaft 1040 and the inner shaft 1030. The balloon 1010 includes a distal portion 1011, a proximal portion 1012, and a central portion 1013. The central portion 1013 is the portion over which the prosthetic heart valve 220 is crimped. The balloon catheter 1000 further includes retention bumpers 1001 and reinforcing members 1003, described in greater detail below.

The portion of the balloon catheter 1000 over which the balloon 1010 is disposed defines a delivery portion 1090 of the balloon catheter 1000. The balloon catheter 1000 further includes an operational portion 1050 configured to receive the prosthetic heart valve 220 crimped thereon. The operational portion 1050 is located between the proximal and distal retention bumpers 901 and corresponds to the central portion 1013 of the balloon 1010.

The balloon catheter 1000 includes retention bumpers 1001 and reinforcing members 1003. The reinforcing members 1003 are cylindrical tubes concentric with the inner shaft 1030 extending over portions of the inner shaft 1030. A respective reinforcing member 1003 is disposed adjacent each retention bumper 1001 extends into the central portion 950. The reinforcing member 1003 adjacent the distal retention bumper 1001 extends proximally of the distal retention bumper 1001. The reinforcing member 1003 adjacent the proximal retention bumper 1001 extends distally of the proximal retention bumper 1001. The balloon 1010 is wrapped over the retention bumpers 1001 to create enlarged balloon portions 1002 having an increased diameter with respect to the remainder of the balloon 1010 when the balloon 1010 is uninflated. Cross-section projections of the enlarged balloon portions 1002 define a protection zone 1085, similar to protection zones discussed above. The proximal and distal retention bumpers 1001 establish the protection zone 1085. The reinforcing members 1003 are of a small enough diameter such that they do not interfere with the wrapping of the balloon 1010 and crimping of the prosthetic heart valve 220. The reinforcing members 1003 extend within the wrapped balloon 1010 and crimped prosthetic heart valve 220. The reinforcing members 1003 define the outside axial ends of a reinforced portion 1080 of the balloon catheter 1000.

The reinforcing members 1003 provide an increased stiffness or rigidity to the axial ends of the reinforced portion 1080 and thereby reinforce the gap between the retention bumpers 1001 and the prosthetic heart valve 220. The reinforcing members 1003 provide an increased stiffness to the balloon catheter 1000 over an extended length as compared to the retention bumpers 1001 alone. The longer length of increased stiffness permits a gradual bending within the longer length and thereby reduces the likelihood of catheter kinking inside or outside of the reinforced portion 1080. The reinforcing members 1003 may extend axially from the retention bumpers 1001 by a distance in the range of 1-8 mm or the range of 2-5 mm.

In embodiments, the reinforcing members 1003 may be manufactured of a material that is more flexible and/or less stiff than a material of the retention bumpers 1001. The difference in stiffnesses between these two parts may create a gradual change from the reinforcing members 1003 to the portion of the balloon catheter 1000 between the reinforcing members 1003. A less abrupt change in stiffness may reduce the likelihood of kinking in the balloon catheter 1000.

In embodiments, the reinforcing members 1003 may be manufactured for radiopacity. Radiopaque filler materials may be included in the polymers, elastomers, etc., that comprise the reinforcing members 1003. Reinforcing members 1003 having increased radiopacity may facilitate imaging (e.g., fluoroscopy) during procedures.

The reinforcing members 1003 are formed via overmolding of the inner shaft 1030. The retention bumpers 1001 are overmolded over the reinforcing members 1003 to secure both the reinforcing members 1003 and the retention bumpers 1001. In further embodiments, the retention bumpers 1001 may be overmolded adjacent to but not overlapping with the reinforcing members 1003. In still further embodiments, one or both of the retention bumpers 1001 and reinforcing members 1003 may be injection molded and then secured to the inner shaft 1030 via bonding, adhesives, or other means.

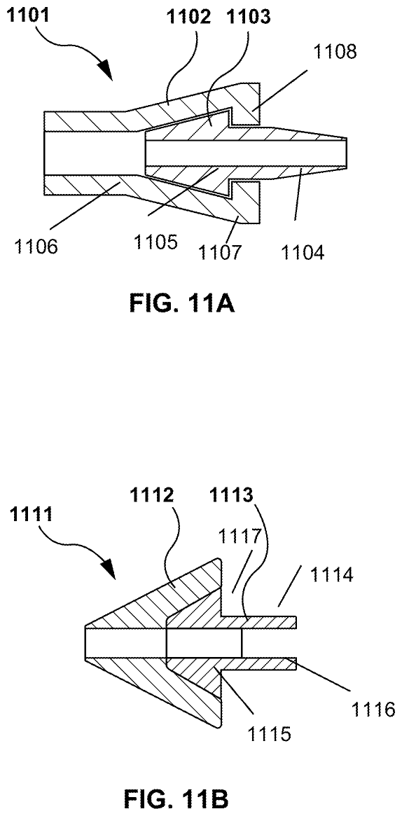

FIGS. 11A-11B illustrate multipart retention bumpers consistent with embodiments hereof. The multipart retention bumpers 1101 and 1111 are configured to provide benefits similar to the retention bumpers 901 and the retention bumper 1001 and reinforcing member 1003 combination.

FIG. 11A illustrates a multipart retention bumper 1101, including an outer bumper 1102 and an inner wedge 1103. During catheter assembly, one of the inner wedge 1103 and outer bumper 1102 of each multipart retention bumper 1101 may be overmolded or otherwise secured to an inner shaft. The non-secured one of the inner wedge 1103 and outer bumper 1102 of each multipart retention bumper 1101 may then be interlocked with the secured portion to form the multipart retention bumper 1101. Multipart retention bumpers 1101 may be employed, for example, because the outer bumper 1102 has a smaller diameter than the assembled multipart retention bumpers 1101. The smaller diameter outer bumper 1102 may facilitate catheter assembly, for example, by more easily fitting through a balloon opening than a full diameter retention bumper.

The inner wedge 1103 includes a reinforcement portion 1104 that extends away from the outer bumper 1102 when the inner wedge 1103 and outer bumper 1102 are interlocked. When employed in a balloon catheter, such as any of those described herein, the reinforcing portion 1104 of each inner wedge 1103 extends axially inward into the central portion of the balloon catheter from the outer bumper 1102. The inner wedge 1103 further includes a support portion 1105. The support portion 1105 is a tapered body with an axial hollow to accommodate a balloon catheter inner shaft. The support portion 1105 has a diameter that gets progressively larger from an apex 1106 at one end of the inner wedge 1103 to a base 1107 in a middle portion of the inner wedge 1103. The inner wedge 1103 diameter abruptly narrows at a shoulder 1108 at the transition between the support portion 1105 and the reinforcement portion 1104. The reinforcement portion 1104 tapers from a base 1109 that meets the support portion 1105 to an apex 1110 at the end of the inner wedge 1103 opposite the apex 1109 of the support portion 1105.

The outer bumper 1102 includes a cylindrical portion 1116, a migration prevention portion 1117, and a locking portion 1118. The cylindrical portion 1116 is a cylinder of material with an axial hollow configured to accommodate an inner shaft. A first end of the cylindrical portion 1106 coincides with a first end of the outer bumper 1102. A second end of the cylindrical portion 1106 meets the migration prevention portion 1117. The migration prevention portion 1117 tapers outwards from the cylindrical portion 1116 to a diameter large enough to function as a retention bumper when interlocked with the support portion 1105 of the inner wedge 1103, i.e., large enough to create a protection zone as described herein. The ends of the migration portion 1117 coinciding with the second end of the outer bumper 1102 meet the locking portion 1118. The locking portion 1118 includes one or more tabs or projections that extend radially inwards. When the outer bumper 1102 and the inner wedge 1103 are interlocked, the tab(s) of the locking portion 1118 snap over or otherwise wrap around the base of the support portion 1105, thereby causing the interlock. The support portion 1105 of the inner wedge 1103 presses outward on the migration prevention portion 1117 of the outer bumper 1102 such that the diameter of the outer bumper 1102 is greater when interlocked with the inner wedge 1103 than when not interlocked with the inner wedge 1103. The reinforcement portion 1104 of the inner wedge 1103 may extend axially from the locking portion 1118 of the outer bumper 1102 by a distance in the range of 1-8 mm or the range of 2-5 mm when the inner wedge 1103 and the outer bumper 1102 are interlocked.

In embodiments, the reinforcement portion 1104 functions similarly to the reinforcement portions 903 of the retention bumpers 901 or to the reinforcement member 1003. The reinforcement portions 1104 extend into the central portion of the balloon catheter and provide reinforcement to the gap between the prosthetic heart valve and the migration prevention portion 1107. The inner wedges 1103 may be manufactured of a material similar to, greater than, or less than the stiffness of the outer bumper 1102 to create a structure that provides an increased bending stiffness to a balloon catheter at the outer axial ends of a central portion, as discussed above, e.g., with respect to FIGS. 9 and 10.

FIG. 11B illustrates a multipart retention bumper 1121, including an outer bumper 1122 and an inner wedge 1123. During catheter assembly, one of the inner wedge 1123 and outer bumper 1122 of each multipart retention bumper 1121 may be overmolded or otherwise secured to an inner shaft. The non-secured one of the inner wedge 1123 and outer bumper 1122 of each multipart retention bumper 1121 may then be interlocked with the secured portion to form the multipart retention bumper 1121. Multipart retention bumpers 1121 may be employed, for example, because the outer bumper 1122 has a smaller diameter than the assembled multipart retention bumpers 1121. The smaller diameter outer bumper 1122 may facilitate catheter assembly, for example, by more easily fitting through a balloon opening than a full diameter retention bumper.

The inner wedge 1123 includes a reinforcement portion 1124 that extends away from the outer bumper 1122 when the inner wedge 1123 and the outer bumper 1122 are interlocked. When employed in a balloon catheter, such as any of those described herein, the reinforcing portion 1124 of each inner wedge 1123 extends axially inward into the central portion of the balloon catheter. The inner wedge 1123 further includes a support portion 1125. The support portion 1125 is a tapered body with an axial hollow to accommodate a balloon catheter inner shaft. The support portion 1125 has a diameter that gets progressively larger from an apex 1126 at one end of the inner wedge 1123 to a base 1127 in the middle of the inner wedge 1123. The inner wedge 1123 diameter abruptly narrows at a shoulder 1128 adjacent the base 1127 of the support portion 1125, i.e., the transition between the support portion 1125 and the reinforcement portion 1124. The reinforcement portion 1124 is generally cylindrical and hollow, extending away from the base of the support portion 1125. The inner wedge 1123 further includes extensions 1129 extending from the reinforcement portion 1124 and disposed circumferentially around a central hollow configured to accommodate an inner shaft. When the inner wedge 1123 is mated with an inner shaft, each extension 1129 extends around a partial circumference of the inner shaft.

The outer bumper 1122 is a tapered body that tapers from an apex at one end to a base at the other end. The tapered body of the outer bumper 1122 surrounds a central hollow configured to accommodate an inner shaft. At the base end of the outer bumper 1122, a tapered hollow 1130, sized and configured to accommodate the support portion 1125 is located. The outer bumper 1122 is configured such that insertion of the support portion 1125 of the inner wedge 1123 causes the tapered body of the outer bumper 1122 to expand in diameter to a diameter large enough to function as a retention bumper, i.e., large enough to create a protection zone as described herein. The reinforcement portion 1124 may extend axially from the support portion 1125 by a distance in the range of 1-8 mm or the range of 2-5 mm.

In embodiments, the reinforcement portion 1124 functions similarly to the reinforcement portions 903 of the retention bumpers 901 or to the reinforcement member 1003. The reinforcement portions 1124 extend into the central portion of the balloon catheter and provide reinforcement to the gap between the prosthetic heart valve and the migration prevention portion 1107. The inner wedges 1123 may be manufactured of a material similar to, greater than, or less than the stiffness of the outer bumper 1122 to create a structure that provides an increased bending stiffness to a balloon catheter at the outer axial ends of a central portion, as discussed above, e.g., with respect to FIGS. 9 and 10.

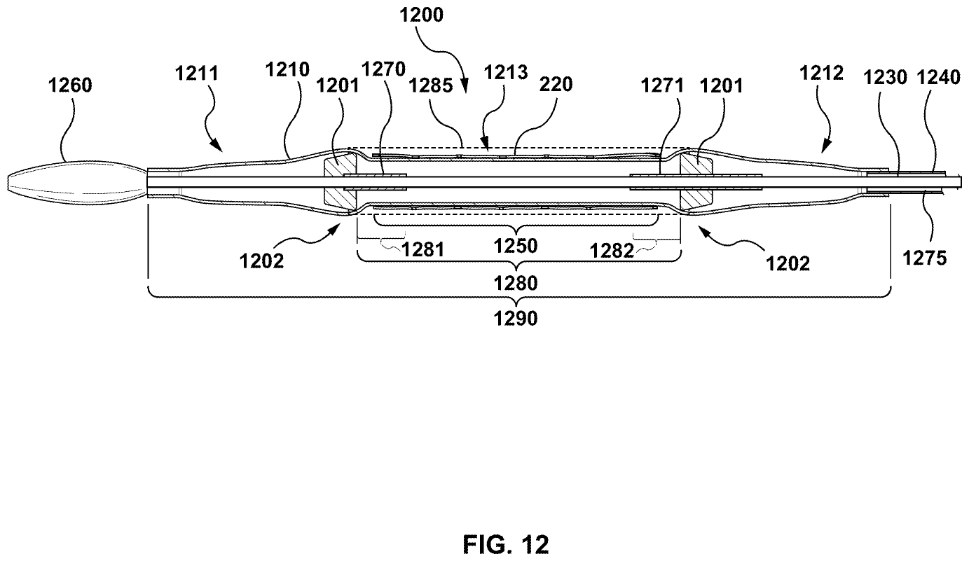

FIG. 12 illustrates a balloon catheter with a proximally extending reinforcement member. FIG. 12 illustrates a balloon catheter 1200 including an outer shaft 1240, an inner shaft 1230, a balloon 1210, and a distal tip 1260. A prosthetic heart valve 220 may be crimped to the balloon 1210 of the balloon catheter 1200. Inflation fluid is delivered to the balloon 1210 via an inflation lumen 1275 disposed between the outer shaft 1240 and the inner shaft 1230. The balloon 1210 includes a distal portion 1211, a proximal portion 1212, and a central portion 1213. The central portion 1213 is the portion over which the prosthetic heart valve 220 is crimped.

The portion of the balloon catheter 1200 over which the balloon 1210 is disposed defines a delivery portion 1290 of the balloon catheter 1200. The balloon catheter 1200 further includes an operational portion 1250 configured to receive the prosthetic heart valve 220 crimped thereon. The operational portion 1250 is located between proximal and distal retention bumpers 1201 and corresponds to the central portion 1213 of the balloon 1210.

The balloon catheter 1200 includes a distal reinforcement member 1270 and a proximal reinforcement member 1271. The distal reinforcement member 1270 extends proximally from within the distal retention bumper 1201. The distal reinforcement member 1270 is a portion of hypotube or other material configured to provide additional stiffness to a first zone 1281 proximally adjacent the distal retention bumper 1201 at the distal end of the central portion 1250. Specific increases to the stiffness or reinforcement of the balloon catheter 1200 in areas adjacent to the retention bumpers 1201 may prevent excessive bending or kinking of the balloon catheter 1200 at the transition between the prosthetic heart valve 220 and the retention bumpers 1201.

The distal reinforcement member 1270 is disposed over and secured to the inner shaft 1230, for example by adhesives, by overmolding, or by reflowing the inner shaft 1230. Additionally or alternatively, the distal reinforcement member 1270 may be secured to the inner shaft 1230 by overmolding the distal retention bumper 1201. The portion of the distal reinforcement member 1270 within the distal retention bumper 1201 may include holes or other openings to permit the overmolded retention bumper 1201 to be secured to the inner shaft 1230 through the hole or openings, thereby also securing the distal reinforcement member 1270 to the distal retention bumper 1201 and the inner shaft 1230. In further embodiments, the distal reinforcement member 1270 may extend through only a portion of the entire length of the distal retention bumper 1201.

The proximal reinforcement member 1271 extends both proximally and distally from within the proximal retention bumper 1201. The proximal reinforcement member 1271 is a portion of hypotube or other material configured to provide additional stiffness to a second zone 1282 at the proximal end of the central portion 1250 and extending proximally of the proximal retention bumper 1201. The additional stiffness provided in the first zone 1281 and the second zone 1282 creates the reinforced portion 1280, which extends proximal of the central portion 1250 and the proximal retention bumper 1201. Specific increases to the stiffness of the balloon catheter 1200 in areas on either side of the proximal retention bumpers 1201 may prevent excessive bending or kinking of the balloon catheter 1200 at the transition between the prosthetic heart valve 220 and the retention bumpers 1201 and the transition between the proximal retention bumper 1201 and the proximal portion of the balloon catheter 1200.

The proximal reinforcement member 1271 is disposed over and secured to the inner shaft 1230, for example by adhesives, by overmolding, or by reflowing the inner shaft 1230. Additionally or alternatively, the proximal reinforcement member 1271 may be secured to the inner shaft 1230 by overmolding of the proximal retention bumper 1201. The portion of the proximal reinforcement member 1271 within the proximal retention bumper 1201 may include holes or other openings to permit the overmolded retention bumper 1201 to be secured to the inner shaft 1230 through the hole or openings, thereby also securing the proximal reinforcement member 1271 to the proximal retention bumper 1201 and the inner shaft 1230.

The balloon 1210 is wrapped over the retention bumpers 1201 to create enlarged balloon portions 1202 having an increased diameter with respect to the remainder of the balloon 1210 when the balloon 1210 is uninflated. Cross-section projections of the enlarged balloon portions 1202 define a protection zone 1285, similar to protection zones discussed above. The proximal and distal retention bumpers 1201 establish the protection zone 1285. The distal reinforcement member 1270 and the proximal reinforcement member 1271 define the axial ends of the reinforced portion 1280.

The distal reinforcement member 1270 and the proximal reinforcement member 1271 provide an increased stiffness or rigidity to the axial ends of the reinforced portion 1280. The distal reinforcement member 1270 and the proximal reinforcement member 1271 provide an increased stiffness to the balloon catheter 1200 over an extended length as compared to the retention bumpers 1201 alone. The longer length of increased stiffness permits a gradual bending within the longer length and thereby reduces the likelihood of catheter kinking outside of the distal reinforcement member 1270 and the proximal reinforcement member 1271.

In further embodiments, the proximal reinforcement member 1271 and the distal reinforcement member 1270 may have reduced stiffness portions and/or a variable stiffness profile. Reduced stiffness portions and/or a variable stiffness profile may be generated through material reductions in the proximal reinforcement member 1271 and/or the distal reinforcement member 1270, similar to those discussed above with respect to FIG. 8C and the reinforcing member 878. Such material reductions may include holes, skives, slots, perforations, or other features that remove material from the proximal reinforcement member 1271 and/or the distal reinforcement member 1270.

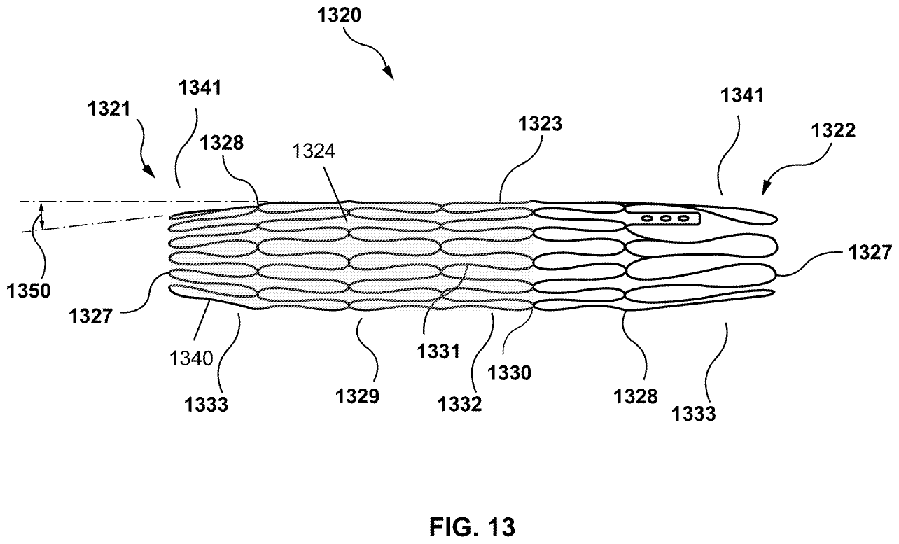

FIG. 13 illustrates a prosthetic heart valve 1320 including a radially expandable stent 1323. The stent 1323 is configured for delivery by a balloon catheter, having a crimped configuration for delivery within a vasculature and an expanded configuration for deployment within a native heart valve. The prosthetic heart valve 1320 includes a stent 1323, graft tissue 1324 disposed thereon, and a valve (not shown). The prosthetic heart valve 1320 has a proximal end 1322 and a distal end 1321. When implanted to replace a native heart valve, the distal end 1321 operates as an outflow end and the proximal end 1322 operates as an inflow end.

The stent 1323 of the prosthetic heart valve 1320 includes a central frame structure 1329 comprising a plurality of struts 1331 and crowns 1330. At each end, each strut 1331 meets another strut 1331 at a crown 1330. A plurality of struts 1331 meeting each other at at a plurality of crowns 1330 forms a zig zag pattern defining a circumference of a row 1132 of the central frame structure 1329. Multiple rows 1332 of struts 1331 and crowns 1330 are connected to one another at the crowns 1330 of each row. Together, the multiple rows 1332 form the length of the central frame structure 1329.

The proximal end 1322 and the distal end 1321 of the stent 1323 are each formed from terminal frame structures 1341. Each terminal frame structure 1341 includes a terminal row 1333 including a plurality of terminal crowns 1327, a plurality of crowns 1330, and a plurality of terminal struts 1340. Each terminal crown 1327 is located at a junction of a pair of terminal struts 1340 and is not connected to any further crowns 1330 or rows 1332. The ends of the terminal struts 1340 opposite the terminal crowns 1327 meet at the crowns 1330. Thus, the terminal rows 1333 connect to the remainder of the rows 1332 at a junction of crowns 1330. The junction of crowns 1330 at which the terminal rows 1333 connect to the remainder of the rows 1332 is an angled crown junction 1328.

The structure and angles of the crowns 1330 and struts 1331 of the central frame structure 1329 may be applied to any suitable prosthetic heart valve frame and structure and are not limited to the illustrations provided herein. Additional examples of prosthetic heart valves to which these features may be applied, as well as additional discussion of the function and delivery of prosthetic heart valves, may be found, for example and not by way of limitation, in U.S. Patent Application Publication No. 2020/0246141 A1, published Aug. 6, 2020, U.S. Patent Application Publication No. 2020/0360134 A1, published Nov. 19, 2020 U.S. Provisional Patent Application No. 62/985,131, filed Mar. 4, 2020, U.S. Provisional Patent Application No. 62/985,124, filed Mar. 4, 2020, and U.S. Provisional Application No. 63/060,378, filed Aug. 3, 2020, each of which is incorporated herein by reference.

As discussed above, the stent 1323 and graft tissue 1324 of the prosthetic heart valve 1320 are configured to expand when deployed via inflation of a balloon. The central frame structure 1329 is approximately cylindrical in shape. The terminal frame structures 1341, attached to the central frame structure 1329 at the angled crown junctions 1328 by the terminal struts 1340, create a crown junction angle 1350 that is substantially non-zero with respect to the central frame structure 1329 at the angled crown junctions 1328. The crown junction angle 1350 may be between 2°-45°, between 10°-40°, between 20°-35°, and/or between 28°-33°. The proximal end 1322 and the distal end 1321 of the stent 1323 may each have a terminal frame structure 1341 joined with the central frame structure 1329 at an angled crown junction 1328 to form a crown-junction angle 1350. The crown junction angle 1350 may be the same at both the proximal end 1322 and the distal end 1321. The crown junction angle 1350 may differ at the proximal end 1322 and the distal end 1321 of the stent 1323. The crown junction angle 1350 is formed between the terminal crowns 1327 and a linear projection of the walls of the central frame structure 1329 at the angled crown junctions 1328. Accordingly, the terminal crowns 1327 and terminal struts 1340 are angled towards a central axis of the prosthetic heart valve 1320.

The prosthetic heart valve 1320 may be non-symmetrical, having different expandable stent 1323 geometry construction at the proximal end 1322 and the distal end 1321. The specific frame structure illustrated in FIG. 13 is by way of example only. Different prosthetic heart valves consistent with embodiments hereof may have different frame geometry.

The prosthetic heart valve 1320 may be formed from stainless steel or other suitable metal, such as nitinol, platinum iridium, cobalt chromium alloys such as MP35N, or various types of polymers or other materials known to those skilled in the art. The shape of the prosthetic heart valve 1320, having inwardly angled terminal frame structures 1341, may be formed during a manufacturing process. The prosthetic heart valve 1320 may be manufactured with inwardly angled terminal frame structures 1341. In further embodiments, the inwardly angled terminal frame structures 1341 may be formed during a crimping process. During balloon catheter assembly, the prosthetic heart valve 1320 is crimped over a balloon of the balloon delivery catheter. A crimping die may be employed to impart the crown junction angle 1350 to the stent 1323 of the prosthetic heart valve 1320 at the angled crown junctions 1328.

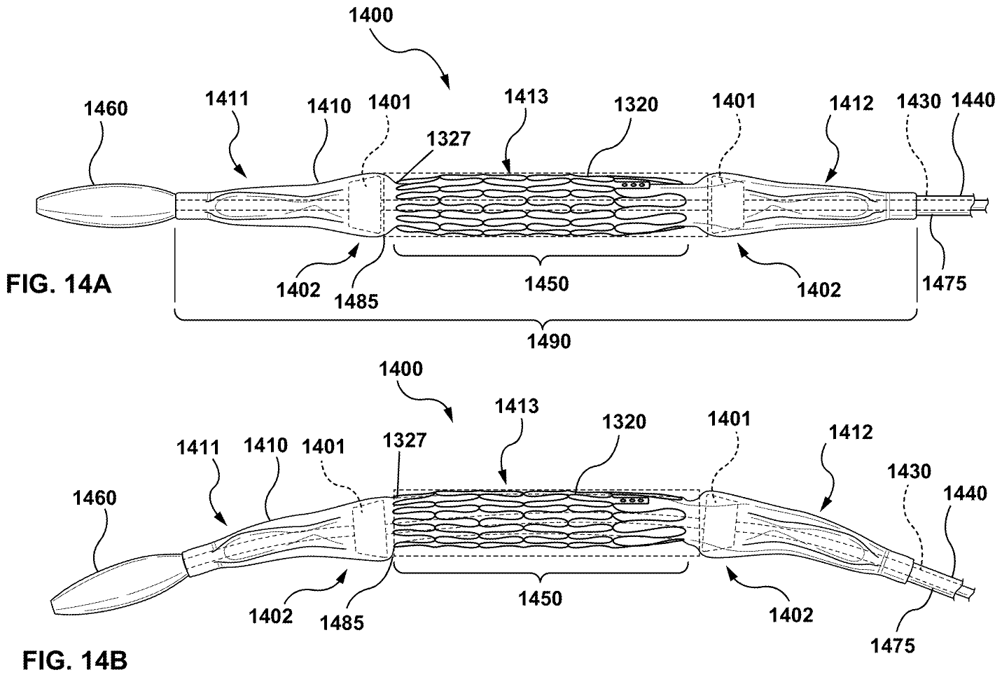

FIGS. 14A and 14B illustrate a balloon catheter with the prosthetic heart valve 1320 crimped thereto. FIG. 14A illustrates the balloon catheter 1400 in a neutral position while FIG. 14B illustrates the balloon catheter 1400 in a curved position. FIGS. 14A and 14B illustrate a balloon catheter 1400 including an outer shaft 1440, an inner shaft 1430, a balloon 1410, and a distal tip 1460. The prosthetic heart valve 1320 is crimped onto the balloon 1410 of the balloon catheter 1400. Inflation fluid is delivered to the balloon 1410 via an inflation lumen 1475 disposed between the outer shaft 1440 and the inner shaft 1430. The balloon 1410 includes a distal portion 1411, a proximal portion 1412, and a central portion 1413. The central portion 1413 is the portion over which the prosthetic heart valve 1320 is crimped.

The portion of the balloon catheter 1400 over which the balloon 1410 is disposed defines a delivery portion 1490 of the balloon catheter 1400. The balloon catheter 1400 further includes an operational portion 1450 configured to receive the prosthetic heart valve 1420 crimped thereto. The operational portion 1450 is located between the proximal and distal retention bumpers 1401 and corresponds to the central portion 1413 of the balloon 1410.

The balloon 1410 is wrapped over the retention bumpers 1401 to create enlarged balloon portions 1402 having an increased diameter with respect to the remainder of the balloon 1410. Cross-section projections of the enlarged balloon portions 1402 define a protection zone 1485, similar to protection zones discussed above. The proximal and distal retention bumpers 1401 establish the protection zone 1485.

During delivery of the prosthetic heart valve 1320, the crown junction angle 1350 assists in minimizing, reducing, or eliminating interference between the terminal crowns 1327 and an introducer sheath through which the balloon catheter 1400 is deployed. Because the terminal frame structures 1341 including the terminal crowns 1327 are angled towards the central axis of the prosthetic heart valve 1320, they remain within the protection zone 1485 created by the enlarged balloon portions 1402 when the balloon catheter 1400 tracks around a curve. The terminal crowns 1327 in the protection zone 1485 remain in radial alignment with the retention bumpers 1401 and therefore do not radially project or protrude from the cross-section projection of the enlarged balloon portions 1402. Additionally, if the terminal crowns 1327 do contact an introducer sheath through which the balloon catheter 1400 is delivered, the inward angle renders the terminal crowns 1327 less likely to catch on the introducer sheath. Accordingly, the crown junction angle 1350 reduces, minimizes, and/or eliminates potential damage to both the introducer sheath and to patient anatomy during prosthetic heart valve delivery.

In embodiments, the crown junction angle 1350 permits the use of retention bumpers having smaller diameters. Due to the reduced diameter at the proximal end 1322 and distal end 1321 of the prosthetic heart valve 1320, a retention bumper having a reduced diameter may be employed. For example, in a design lacking the crown junction angle 1350, wherein the terminal crowns 1327 extend in parallel to the central frame structure 1329, a retention bumper having a diameter of 8.5 mm may be required. When combined with the prosthetic heart valve 1320 including the crown junction angle 1350, retention bumpers of 7.0-7.5 mms may be used. Accordingly, retention bumpers reduced in size by approximately 13%-22% may be used with the prosthetic heart valve 1320 having inwardly angled terminal crowns 1327.

In embodiments, the crown junction angle 1350 provides additional strength to the prosthetic heart valve 1320 and may reduce, minimize, or eliminate “fish-mouthing.” Fish-mouthing refers to the tendency of some prosthetic heart valves to develop an oval profile when tracking around curves. The oval profile causes the crowns of these valves to extend or jut out along the major axis of the oval, further than they would if the valve retained a circular profile. The crown junction angle 1350 of the prosthetic heart valve 1320 directs the terminal crowns 1327 inward towards a central axis of the prosthetic heart valve 1320. This creates additional structure in a different plane than the remainder of the prosthetic heart valve 1320. The additional structure provided by the terminal crowns 1327 angled inward may provide support to the prosthetic heart valve 1320 and resistance against fish-mouthing.

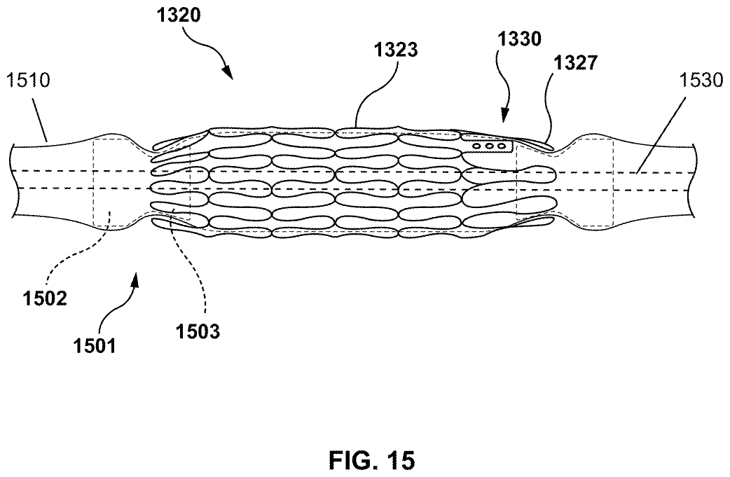

FIG. 15 illustrates a portion of a balloon catheter featuring retention bumpers configured for use with the prosthetic heart valve 1320 according to embodiments hereof. The retention bumpers 1501 are configured with a shape that conforms to that of the prosthetic heart valve 1320. Specifically, the retention bumpers 1501 include a retention portion 1502 and a support portion 1503. The retention bumper 1501 is configured with a central hollow to accommodate an inner shaft 1530 of a balloon catheter.

The retention portion 1502 has radial symmetry. The retention portion 1502 is generally cylindrical and is configured with a diameter sufficient for maintaining the axial position of the prosthetic heart valve 1320 when the retention bumper 1501 is incorporated into a balloon catheter over which the prosthetic heart valve 1320 is crimped, as discussed herein. Due to the inwardly angled terminal crowns 1327 of the prosthetic heart valve 1320, the diameter of the retention portion 1502 may be smaller, e.g., approximately 13%-22% smaller, than the diameter of a retention bumper for a purely cylindrical prosthetic heart valve.

The support portion 1503 has radial symmetry. The support portion 1503 is a tapered body that tapers from a larger diameter at an end opposite the retention portion 1502 to a smaller diameter at an end joining with the retention portion 1502. The taper angle of the support portion 1503 is selected to correspond to the angle of the crown junction angle 1350. For example, the taper angle of the support portion 1503 may be selected to be the same as the crown junction angle 1350. In another example, the taper angle may be selected such that, when the retention bumper 1501 is wrapped with a balloon of a balloon catheter, the enlarged balloon portion corresponding to the taper is at approximately the same angle as the crown junction angle 1350. Accordingly, when the balloon 1510 is secured and wrapped over the balloon the retention bumpers 1501, the shape of the wrapped balloon approximates that of the prosthetic heart valve 1320. Thus, when the prosthetic heart valve 1320 is crimped to the balloon 1510 wrapped over the retention bumpers 1501, the support portion 1503 of the retention bumpers 1501 provides support to the ends of the crimped prosthetic heart valve 1320 while the central structure 1329 of the prosthetic heart valve 1320 is supported by the central area of the balloon 1510.

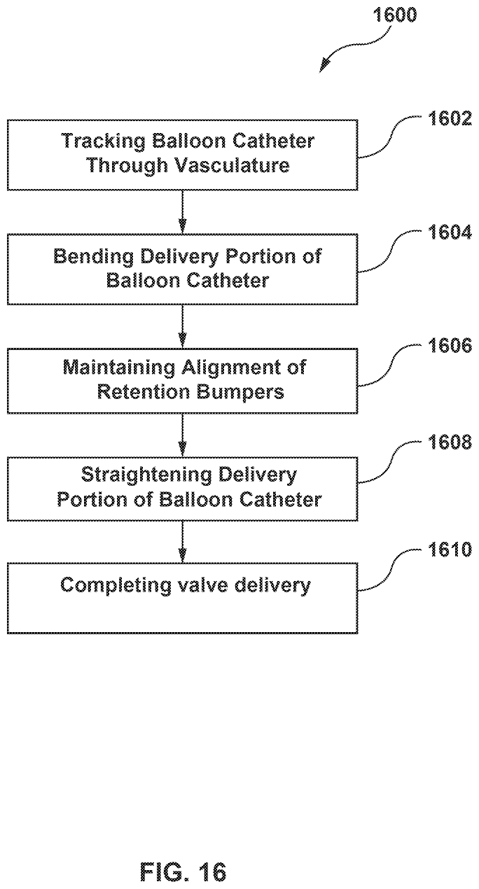

FIG. 16 is a flow chart of a method 1600 of balloon deployment of a prosthetic heart valve consistent with embodiments described herein. The devices and structures described herein reduce or prevent prosthetic heart valve protrusion during balloon delivery through curved vascular pathways. Methods of delivering a prosthetic heart valve to a determined location may be carried out it with any of the embodiments described herein, and with any combination of the embodiments described herein.

In an operation 1602, a balloon catheter is manipulated to navigate the balloon catheter delivery portion to a prosthetic heart valve deployment site. The balloon catheter is navigated through a procedural catheter, a guide catheter, and/or an introducer catheter to deliver the prosthetic heart valve to the site for deployment of the prosthetic heart valve.

In an operation 1604, the delivery portion of the balloon catheter is subject to bending. The delivery portion of the balloon catheter is subject to bending when tracked through a portion of the procedural, guide, or introducer catheter that is itself bent. For example, the procedural, guide, or introducer catheter may be bent so as to clear the aortic arch.

In an operation 1606, a reinforcing member of the balloon catheter operates to maintain alignment of the prosthetic heart valve and the retention bumpers. The delivery portion of the catheter bends as it tracks through the bent portion of the procedural, guide, introducer sheath, and/or patient vasculature. The reinforcing member operates by providing an increased rigidity to the balloon catheter in the reinforced portion, which overlaps with the operational portion, where the prosthetic heart valve is located. The increased rigidity causes greater bending in the delivery portion outside of the reinforced portion. The relative straightness of the balloon catheter in the reinforced portion causes the prosthetic heart valve to remain aligned with the retention bumpers such that it does not extend or protrude beyond the diameter of the retention bumpers. The prosthetic heart valve remains within the protection zone established by the retention bumpers. Accordingly, damage to the introducer sheath and/or patient vasculature or prosthetic heart valve is avoided.

In an operation 1608, the delivery portion of the balloon catheter is straightened after passing through the curved portion of the introducer sheath and/or patient vasculature. After passing through the curved portion of the introducer sheath and/or patient vasculature, the delivery portion of the catheter straightens out with the prosthetic heart valve remaining in alignment with the retention bumpers so that delivery and deployment may continue.

In an operation 1610, the balloon catheter completes traversal of the vasculature and delivers the prosthetic heart valve to a deployment site. After delivery to the deployment site, the prosthetic heart valve is deployed through balloon inflation. Subsequent to deployment, the balloon is deflated and the balloon catheter is withdrawn, leaving the prosthetic heart valve in a deployed position.

The foregoing description has been presented for purposes of illustration and enablement and is not intended to be exhaustive or to limit the invention to the precise form disclosed. Other modifications and variations are possible in light of the above teachings. The embodiments and examples were chosen and described in order to best explain the principles of the invention and its practical application and to thereby enable others skilled in the art to best utilize the invention in various embodiments and various modifications as are suited to the particular use contemplated. It is intended that the appended claims be construed to include other alternative embodiments of the invention.

1. A balloon catheter for deploying a prosthetic valve via balloon inflation, comprising:
an inner shaft;
an outer shaft surrounding the inner shaft;
a balloon disposed at a distal end of the outer shaft defining a delivery portion of the balloon catheter and having an operational portion configured to receive the prosthetic valve;
a distal retention bumper secured to the inner shaft distal of the operational portion; and
a reinforcing member configured to reinforce a portion of the balloon catheter between the proximal retention bumper and the distal retention bumper, wherein the reinforcing member causes an increase in bending stiffness of the balloon catheter at a reinforced portion.

2. The balloon catheter of claim 1, further comprising a proximal retention bumper secured to the inner shaft proximal of the operational portion.

3. The balloon catheter of claim 1, wherein the reinforcing member includes a first reinforcing member and a second reinforcing member.

4. The balloon catheter of claim 3, wherein the first reinforcing member extends distally of a proximal retention bumper secured to the inner shaft proximal of the operational portion and the second reinforcing member extends proximally of the distal retention bumper.

5. The balloon catheter of claim 4, wherein the first reinforcing member further extends proximally of the proximal retention bumper.

6. The balloon catheter of claim 1,
wherein the increase in bending stiffness causes the reinforced portion to remain straighter than remaining portions of the delivery portion of the balloon catheter during curvature of the delivery portion.

7. The balloon catheter of claim 2, wherein the reinforcing member has a first reduced stiffness portion distal of the distal retention bumper and a second reduced stiffness portion proximal of the proximal retention bumper.

8. The balloon catheter of claim 7, wherein the reinforcing member includes a hypotube and wherein the first reduced stiffness portion and the second reduced stiffness portion are laser cut portions of the hypotube.

9. The balloon catheter of claim 1, wherein the reinforcing member includes a tube disposed over the inner shaft over a length of the reinforced portion.

10. The balloon catheter of claim 3, wherein the first reinforcing member is formed integrally with a proximal retention bumper secured to the inner shaft proximal of the operational portion and the second reinforcing member is formed integrally with the distal retention bumper.

11. The balloon catheter of claim 1, wherein the reinforcing member is configured with a variable stiffness profile.

12. The balloon catheter of claim 1, wherein the reinforcing member is a stiffened portion of the inner shaft.

13. The balloon catheter of claim 3, wherein the first reinforcing member is formed from a reinforcing portion of a proximal multipart retention bumper and the second reinforcing member is formed from a reinforcing portion of a distal multipart retention bumper.

14. The balloon catheter of claim 1, wherein the reinforced portion is configured such that the prosthetic valve remains in radial alignment with the distal retention bumper during curvature of the delivery portion.

15. The balloon catheter of claim 3, wherein the first reinforcing member is located adjacent a proximal retention bumper and the second reinforcing member is located adjacent the distal retention bumper.

16. The balloon catheter of claim 1, wherein operational portion is configured to receive the prosthetic valve crimped thereon.

17. A method of deploying a prosthetic valve via a balloon catheter, comprising:
inserting the balloon catheter into an introducer sheath for delivery to a deployment site within vasculature of a patient, the balloon catheter including an inner shaft, an outer shaft surrounding the inner shaft, a balloon disposed at a distal end of the outer shaft, the prosthetic valve being disposed at an operational portion of the balloon catheter, a distal retention bumper secured to the inner shaft distal of the operational portion; and a reinforcing member defining a reinforced portion configured to cause an increase in bending stiffness of the balloon catheter at the reinforced portion;
advancing the balloon catheter through a bend in the vasculature of the patient; and
maintaining an alignment of the prosthetic valve, the distal retention bumper, and the proximal retention bumper to prevent protrusion of the prosthetic valve beyond a diameter of the distal retention bumper.

18. The method of claim 17, wherein advancing the balloon catheter through the bend in the vasculature of the patient includes maintaining the reinforced portion in a straighter configuration than remaining portions of a delivery portion of the balloon catheter during curvature of the delivery portion.

19. The method of claim 17, wherein advancing the balloon catheter through the bend in the vasculature of the patient includes causing a first flex point proximal to the reinforced portion and a second flex point distal to the reinforced portion.

20. The method of claim 17, wherein advancing the balloon catheter through the bend in the vasculature of the patient includes maintaining the prosthetic valve in alignment with the distal retention bumper during curvature of the delivery portion.

21-53. (canceled)
